(19) United States
(12) Patent Application Publication
(10) Pub. No.: US 2013/0022646 A1
(43) Pub. Date: Jan. 24, 2013

(54) Controlled Release Formulations of Opioids

(71) Applicants: Edward M. Rudnic, Potomac, MD (US); Michael Vachon, Quebec (CA); Gary W. Pace, La Jolla (CA)

(72) Inventors: Edward M. Rudnic, Potomac, MD (US); Michael Vachon, Quebec (CA); Gary W. Pace, La Jolla (CA)

(21) Appl. No.: 13/442,849

(22) Filed: Apr. 9, 2012

Related U.S. Application Data
(63) Continuation-in-part of application No. 13/024,319, filed on Feb. 9, 2011.
Application No. 13/024,319 is a continuation-in-part of application No. 13/400,065, filed on Feb. 18, 2012.
Application No. 13/400,065 is a continuation-in-part of application No. 13/400,004, filed on Feb. 17, 2012.
(60) Provisional application No. 61/302,698, filed on Feb. 9, 2010.
(60) Provisional application No. 61/386,277, filed on Sep. 24, 2010.
(60) Provisional application No. 61/443,966, filed on Feb. 17, 2011.

(51) Int. Cl. A61K 31/485; A61P 25/04; A61K 9/00

(52) U.S. Cl. 424/400; 514/282

(57) ABSTRACT

Pharmaceutical formulations containing opioid components that each has a release profile. The components may provide immediate or controlled release of the opioid. The invention is also directed to methods of controlling release of one or more opioid compounds and methods of treating pain.

### CROSS-REFERENCES TO RELATED APPLICATIONS

This application is a continuation-in-part of U.S. application Ser. No. 13/024,319, filed on Feb. 9, 2011, which claims priority to U.S. provisional application Ser. No. 61/302,698, filed Feb. 9, 2010, and to U.S. provisional application Ser. No. 61/386,277, filed Sep. 24, 2010. This application is also a continuation-in-part of U.S. application Ser. No. 13/400,065, filed on Feb. 18, 2012, which is a continuation-in-part of Ser. No. 13/400,004, filed on Feb. 17, 2012, which claims priority to U.S. provisional application Ser. No. 61/443,966, filed on Feb. 17, 2011. The entirety of each of these applications is incorporated herein by reference.

### FIELD OF THE INVENTION

The invention is directed to pharmaceutical formulations comprising opioid components that each has a release profile. The components may provide immediate or controlled release of the opioid. The invention is also directed to methods of controlling release of one or more opioid compounds and methods of treating pain.

### BACKGROUND OF THE INVENTION

Opioids are a class of pain-relieving prescription medications frequently used in the treatment of a variety of acute and chronic, moderate to severe, pain. However, opioids can be rapidly absorbed and systemically excreted by the body through metabolic inactivation. In order to treat patients, especially those in severe pain, administration of opioids often requires careful dosing at frequent intervals to maintain effective steady state blood levels of the opioid, and thereby provide consistent analgesia. Otherwise, blood levels of the opioid can oscillate, resulting in poor and inconsistent pain relief.

These difficulties associated with the administration of opioids suggests a need to develop an opioid therapy that can, following administration, maintain consistent levels of opioid in the blood and avoid oscillations in pain relief.

### SUMMARY OF THE INVENTION

The invention relates to pharmaceutical formulations for treating pain that comprise components containing opioid compounds. The invention also relates to methods of controlling release of one or more opioid compounds and methods of treating pain.

The pharmaceutical formulations of the invention may comprise one or more components having one or more release profiles, in which at least one of the components comprise a compound having opioid receptor agonist activity. In embodiments wherein there is more than one component, the components may have the same release profile, or the components may have different release profiles.

In some embodiments, the compounds having opioid receptor agonist activity may have agonist activity toward the mu (“μ,” morphine receptor), sigma (“σ,” the phencyclidine receptor), kappa (“κ,” the ketocyclazocine receptor) or delta (“δ,” the endorphinlenkephalin receptor) opioid receptors. Such compounds may include, among others, morphine, codeine, hydromorphone, hydrocodone, oxycodone, dihydrocodeine, dihydromorphine, oxymorphone, mixtures thereof, or salts thereof. In certain embodiments, a component may comprise two opioid compounds in varying ratios. In particular embodiments, a component may comprise morphine and oxycodone, or salts thereof, in about a 3:2 ratio by weight.

In some embodiments, the components may have an immediate release profile or a controlled release profile.

In certain embodiments, the formulation may comprise one or more additional components, such as at least two, at least three, at least four, or at least five components. In some embodiments, the one or more additional components may comprise one or more active agents. In some embodiments, the one or more active agents may be compounds having opioid receptor agonist activity. In some embodiments, the one or more active agents may be one or more non-opioid analgesic compound(s), or a mixture of one or more non-opioid analgesic compound(s) and one or more compound(s) with opioid receptor agonist activity, or pharmaceutically acceptable salts, esters or prodrugs thereof. In certain embodiments, the one or more active agents may be one or more hybrid opioid compound(s), or a mixture of one or more hybrid opioid compound(s) and one or more compound(s) with opioid receptor agonist activity, or pharmaceutically acceptable salts, esters or prodrugs thereof.

In embodiments of the invention, the pharmaceutical formulation may comprise a controlled release component, wherein the controlled release component comprises one or more cores. In additional embodiments, the controlled release component comprises oxycodone. In embodiments of the invention, the pharmaceutical formulation comprises a steady state plasma concentration profile of the oxycodcone having a fluctuation index of about 90% or less. In some embodiments, the pharmaceutical formulation comprises an AUC_{ss,τ}, such that when the pharmaceutical formulation contains about 20 mg of oxycodone and the dosing interval is 12 hours, AUC_{ss,τ} of oxycodone is about 100 ng*h/mL to about 550 ng*h/mL. In other embodiments, when the pharmaceutical formulation contains about 20 mg of oxycodone and the dosing interval is 12 hours, C_{ss,max} of oxycodone is about 10 ng/mL to about 50 ng/mL. In some embodiments, when the pharmaceutical formulation contains about 20 mg of oxycodone and the dosing interval is 12 hours, t_{ss,max} of oxycodone is about 1 hour to about 10 hours.

In other embodiments, when the pharmaceutical formulation contains a different total dose of oxycodone than about 20 mg, AUC_{ss,τ} of oxycodone at the different total dose is proportional to AUC_{ss,τ} of oxycodone at about 20 mg. In particular embodiments, when the pharmaceutical formulation contains a different total dose of oxycodone than about 20 mg, C_{ss,max} of oxycodone at the different total dose is proportional to C_{ss,max} of oxycodone at about 20 mg.

In additional embodiments, the pharmaceutical formulation contains about 20 mg of oxycodone, and the AUCₜ of oxycodone is about 70 ng*h/mL to about 352 ng*h/mL following a single administration of the pharmaceutical formulation. In other embodiments, when the pharmaceutical formulation comprises about 20 mg of oxycodone, Cₘₐₓ of oxycodone is about 5 ng/mL to about 15 ng/mL following a single administration of the pharmaceutical formulation. In some embodiments, when the pharmaceutical formulation contains about 20 mg of oxycodone, the tₘₐₓ of oxycodone is about 4 hours to about 24 hours following a single administration of the pharmaceutical formulation.

In other embodiments, when the pharmaceutical formulation contains a different total dose of oxycodone than about 20 mg, AUCₜ of oxycodone at the different total dose is proportional to AUCₜ of oxycodone at about 20 mg. In particular embodiments, when the pharmaceutical formulation contains a different total dose of oxycodone than about 20 mg, Cₘₐₓ of oxycodone at the different total dose is proportional to C ₘₐₓ of oxycodone at about 20 mg.

In additional embodiments, the controlled release component comprises morphine. In embodiments of the invention, the pharmaceutical formulation comprises a steady state plasma concentration profile of the morphine having a fluctuation index of about 90% or less. In some embodiments, the pharmaceutical formulation comprises an AUC_{ss,τ}, such that when the pharmaceutical formulation contains about 30 mg of morphine and the dosing interval is 12 hours, AUC_{ss,τ} of morphine is about 60 ng*h/mL to about 240 ng*h/mL. In other embodiments, when the pharmaceutical formulation contains about 30 mg of morphine and the dosing interval is 12 hours, C_{ss,max} of morphine is about 8 ng/mL to about 29 ng/mL. In some embodiments, when the pharmaceutical formulation contains about 30 mg of morphine and the dosing interval is 12 hours, t_{ss,max} of morphine is about 1 hour to about 5 hours.

In other embodiments, when the pharmaceutical formulation contains a different total dose of morphine than about 30 mg, AUC_{ss,τ} of morphine at the different total dose is proportional to AUC_{ss,τ} of morphine at about 30 mg. In particular embodiments, when the pharmaceutical formulation contains a different total dose of morphine than about 30 mg, C_{ss, max} of morphine at the different total dose is proportional to C_{ss, max} of morphine at about 30 mg.

In additional embodiments, the pharmaceutical formulation contains about 30 mg of morphine, and the AUCₜ of morphine is about 60 ng*h/mL to about 433 ng*h/mL following a single administration of the pharmaceutical formulation. In other embodiments, when the pharmaceutical formulation comprises about 30 mg of morphine, Cₘₐₓ of morphine is about 1 ng/mL to about 11 ng/mL following a single administration of the pharmaceutical formulation. In some embodiments, when the pharmaceutical formulation contains about 30 mg of morphine, the tₘₐₓ of morphine is about 3 hours to about 25 hours following a single administration of the pharmaceutical formulation.

In other embodiments, when the pharmaceutical formulation contains a different total dose of morphine than about 30 mg, AUCₜ of morphine at the different total dose is proportional to AUCₜ of morphine at about 30 mg. In particular embodiments, when the pharmaceutical formulation contains a different total dose of morphine than about 30 mg, Cₘₐₓ of morphine at the different total dose is proportional to Cₘₐₓ of morphine at about 30 mg.

In another embodiment, the controlled release component comprises oxycodone hydrochloride and morphine sulfate. In a further embodiment when the pharmaceutical formulation comprises a total dose of about 20 mg oxycodone hydrochloride and about 30 mg morphine sulfate, the Cₘₐₓ of oxycodone is about 5 ng/mL to about 15 ng/mL and the Cₘₐₓ of morphine is about 1 ng/mL to about 11 ng/mL following a single administration of the pharmaceutical formulation. In other embodiments, when the pharmaceutical formulation comprises a total dose of about 20 mg oxycodone hydrochloride and about 30 mg morphine sulfate, the tₘₐₓ is about 4 hours to about 24 hours for oxycodone and about 3 hours to about 25 hours for morphine following a single administration of the pharmaceutical formulation. In yet other embodiments, when the pharmaceutical formulation comprises a total dose of about 20 mg oxycodone hydrochloride and about 30 mg morphine sulfate, the AUCₜ is about 70 ng*h/mL to about 352 ng*h/mL for oxycodone and about 60 ng*h/mL to about 433 ng*h/mL for morphine following a single administration of the pharmaceutical formulation.

In certain embodiments, the opioid is selected from the group consisting of morphine, codeine, hydromorphone, hydrocodone, oxycodone, dihydrocodeine, dihydromorphine, oxymorphone, mixtures thereof, and salts thereof. In particular embodiments, the opioid is oxycodone, morphine, mixtures thereof or a salt thereof.

In certain embodiments, the pharmaceutical formulation is in the form of a tablet or capsule. In some embodiments, the pharmaceutical formulation is in the form of a tablet.

In certain embodiments, the controlled release opioid component is in a form selected from the group consisting of pellets, beads, beadlets, granules, powder, or a combination thereof. In some embodiments, the controlled release opioid component is in the form of a beadlet.

In embodiments of the invention, the pharmaceutical formulation further comprises an abuse deterrent component. In some embodiments, the abuse deterrent component comprises a core comprising one or more materials that are both hydrophilic and hydrophobic, and optionally a coating. In some embodiments, the material that is both hydrophilic and hydrophobic is selected from the group consisting of polyacrylic acid, acrylic acid cross-linked with allyl ethers of polyalcohols, hydroxypropyl cellulose, hydroxypropyl methylcellulose:hydroxypropyl cellulose mixture, polyvinylpyrrolidone, polyethylene oxide, methylcellulose, xanthan gum, guar gum, polyethylene glycol, methacrylic acid copolymer, colloidal silicon dioxide, cellulose gum, starch, sodium starch glycolate, sodium alginate, and combinations thereof. In certain embodiments, the material that is both hydrophilic and hydrophobic is acrylic acid cross-linked with allyl ethers of polyalcohols. In some embodiments, the acrylic acid cross-linked with allyl ethers of polyalcohols is a carbomer, such a Carbopol. In some embodiments, the abuse deterrent component further comprises an alkalizing agent. In certain embodiments, the alkalizing agent is meglumine. In certain embodiments, the abuse deterrent component is in a form selected from the group consisting of pellets, beads, beadlets, granules, powder, or a combination thereof.

In some embodiments, the pharmaceutical formulation further comprises one or more fillers or diluents, one or more hydrophilic polymers, one or more disintegrants, and one or more lubricants. In certain embodiments, the one or more fillers or diluents comprises microcrystalline cellulose, such as microcrystalline cellulose PH102 and/or PH200. In some embodiments, the one or more hydrophilic polymers comprises a carbomer, such as Carbopol 971P. In embodiments of the invention, the one or more disintegrants comprises croscarmellose sodium, and the one or more lubricants comprises magnesium stearate.

The method for controlling release of one or more compounds having opioid receptor agonist activity for absorption in a human comprises administering a pharmaceutical formulation comprising one or more components, such that the one or more opioid components comprise one or more release profiles, and at least one of the opioid components is a controlled release opioid component comprising an opioid. In certain embodiments, the pharmaceutical formulation administered to the human is in accordance with the pharmaceutical formulations of the invention.

The method of treating pain in a human comprises administering a pharmaceutical formulation comprising one or more opioid components, such that the one or more opioid components comprise one or more release profiles, and at least one of the opioid components is a controlled release opioid component comprising an opioid. In certain embodiments, the pharmaceutical formulation administered to the human is in accordance with the pharmaceutical formulations of the invention.

### BRIEF DESCRIPTION OF DRAWINGS

FIGS. 1a and 1b provide schematic images of two embodiments of opioid formulations of the present invention.

FIG. 2 provides a target release profile for oxycodone coated pellets used in the opioid formulations of the present invention.

FIG. 3 provides a target release profile for morphine coated pellets used in the opioid formulations of the present invention.

FIG. 4 provides a target release profile for oxycodone granulation coated only with Eudragit L30D-55 used in the opioid formulations of the present invention.

FIG. 5 provides a target release profile for total oxycodone release in the opioid formulations of the present invention.

FIG. 6 provides a target release profile for total oxycodone and morphine release used in the dual-opioid coated tablets of the present invention.

FIG. 7 provides a schematic demonstrating the methods used in producing the oxycodone granules used in the present invention.

FIG. 8 provides a schematic demonstrating the methods used in producing the oxycodone core pellets used in the present invention.

FIG. 9 provides a schematic demonstrating the methods used in producing the morphine core pellets used in the present invention.

FIG. 10 provides a schematic demonstrating the methods used in coating the either morphine or oxycodone core pellets used in the present invention.

FIG. 11 provides a schematic demonstrating the methods used in producing the dual opioid coated tablets used in the present invention.

FIG. 12 provides a flow diagram for preparing extended release intermediate oxycodone pellets used in the clinical study (Example 2).

FIG. 13 provides an oxycodone plasma concentration profile of two opioid formulations of the present invention (Formulation A and Formulation B) and a Reference Formulation (MS Contin® 30 mg (morphine CR) co-administered with OxyContin® 20 mg (oxycodone CR)) through 72 hours after treatment.

FIG. 14 provides an oxycodone plasma concentration profile of two opioid formulations of the present invention (Formulation A and Formulation B) and a Reference Formulation (MS Contin® 30 mg (morphine CR) co-administered with OxyContin® 20 mg (oxycodone CR)) through 24 hours after treatment.

FIG. 15 provides a projected oxycodone plasma profile from administration of multiple doses at 12 hour intervals of an opioid formulation of the present invention.

FIG. 16 provides a projected oxycodone plasma profile from administration of multiple doses of an opioid formulation of the present invention having different dosing strengths.

FIG. 17 provides a projected oxycodone plasma profile from administration of multiple doses at 12 hour intervals of an opioid composite formulation (immediate release +controlled release) of the present invention.

FIG. 18 provides a projected oxycodone plasma profile from administration of multiple doses of an opioid composite formulation (immediate release +controlled release) of the present invention having different dosing strengths.

FIG. 19 provides a release profile of morphine sulfate from coated beadlets containing morphine sulfate and oxycodone hydrochloride using Ammonio Methacrylate Copolymer Type B (RS) and Type A (RL) coating ratios of (a) RS/RL=90/10 and (b) RS/RL=80/20, at various % coating levels.

FIG. 20 provides a release profile of oxycodone hydrochloride from coated beadlets containing morphine sulfate and oxycodone hydrochloride using Ammonio Methacrylate Copolymer Type B (RS) and Type A (RL) coating ratios of (a) RS/RL=90/10 and (b) RS/RL=80/20, at various % coating levels.

FIG. 21 provides a release profile of morphine sulfate in enteric coated tablets (using 50% coated beadlets of Ammonio Methacrylate Copolymer Type B (RS) and Type A (RL) in a ratio of 90/10) at various % enteric coating levels.

FIG. 22 provides a release profile of oxycodone hydrochloride in enteric coated tablets (using 50% coated beadlets of Ammonio Methacrylate Copolymer Type B (RS) and Type A (RL) in a ratio of 90/10) at various % enteric coating levels.

FIG. 23 provides a release profile for morphine sulfate in enteric coated tablets (10% and 15% coating level) at low, mid or high hardness levels.

FIG. 24 provides a release profile for oxycodone hydrochloride in enteric coated tablets (10% and 15% coating level) at low, mid or high hardness levels. FIG. 25 provides a release profile of oxycodone hydrochloride from coated beadlets containing oxycodone hydrochloride using Ammonio Methacrylate Copolymer Type B (RS) and Type A (RL) coating ratios of RS/RL=85/15 at various % coating levels.

FIG. 26 provides a release profile of oxycodone hydrochloride from coated beadlets containing oxycodone hydrochloride using Ammonio Methacrylate Copolymer Type B (RS) and Type A (RL) coating ratios of RS/RL=80/20 at various % coating levels.

FIG. 27 provides a release profile of oxycodone hydrochloride from coated beadlets containing oxycodone hydrochloride using Ammonio Methacrylate Copolymer Type B (RS) and Type A (RL) coating ratios of RS/RL=80/20 and RS/RL=85/15 at various % coating levels.

FIG. 28 provides a schematic of the formation of MoxDuo CR tablets.

FIG. 29 provides morphine plasma concentrations following single dose administration of MoxDuo CR beadlets (30:20 mg) and MoxDuo CR tablet (30:20 mg), and MS Contin/OxyContin.

FIG. 30 provides oxycodone plasma concentrations following single dose administration of MoxDuo CR beadlets (30:20 mg) and MoxDuo CR tablet (30:20 mg), and MS Contin/OxyContin.

FIG. 31 provides mean morphine plasma concentrations following steady-state administration of MoxDuo CR tablets (30:20 mg).

FIG. 32 provides mean morphine plasma concentrations following steady-state administration of MoxDuo CR tablets (30:20 mg).

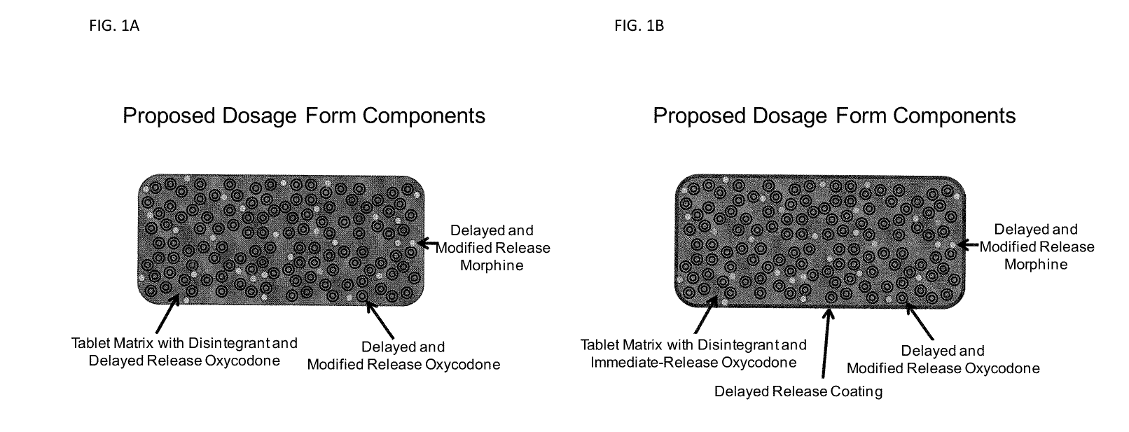

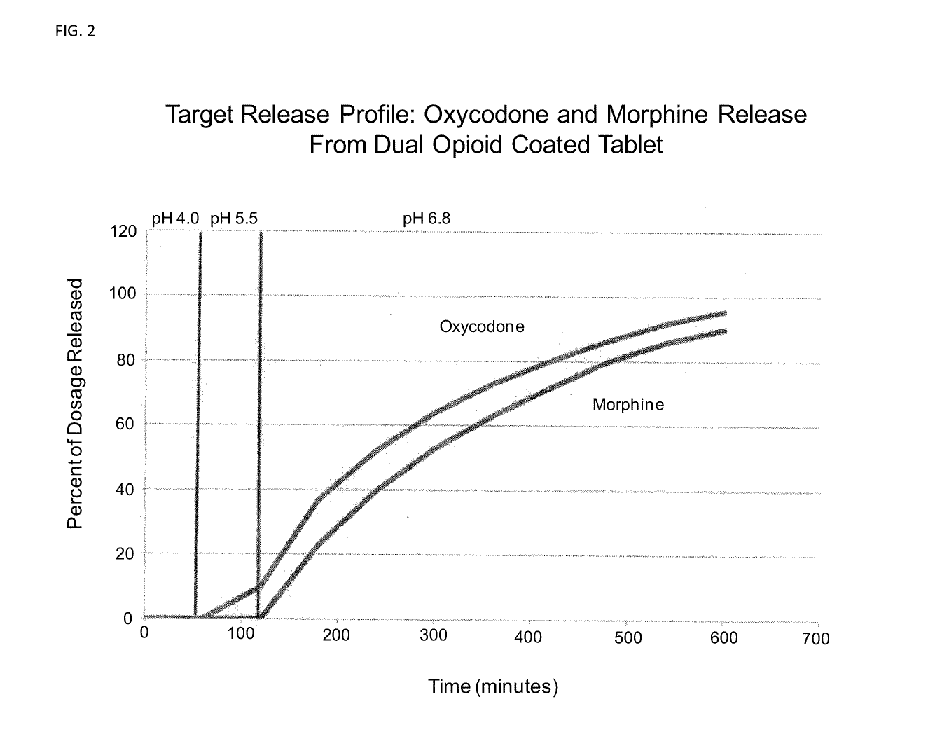

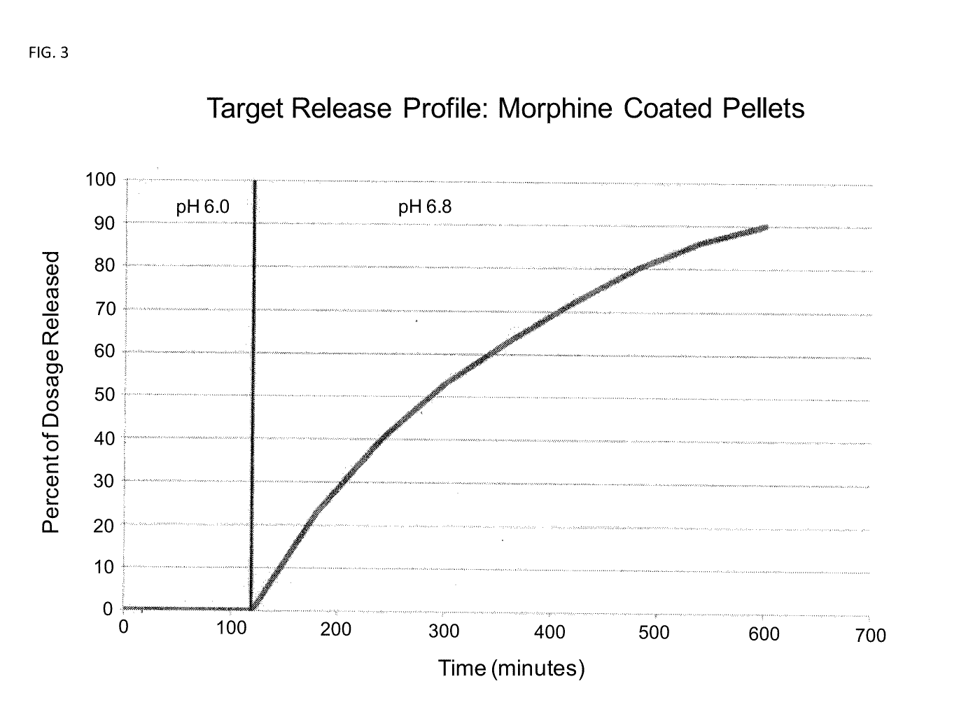

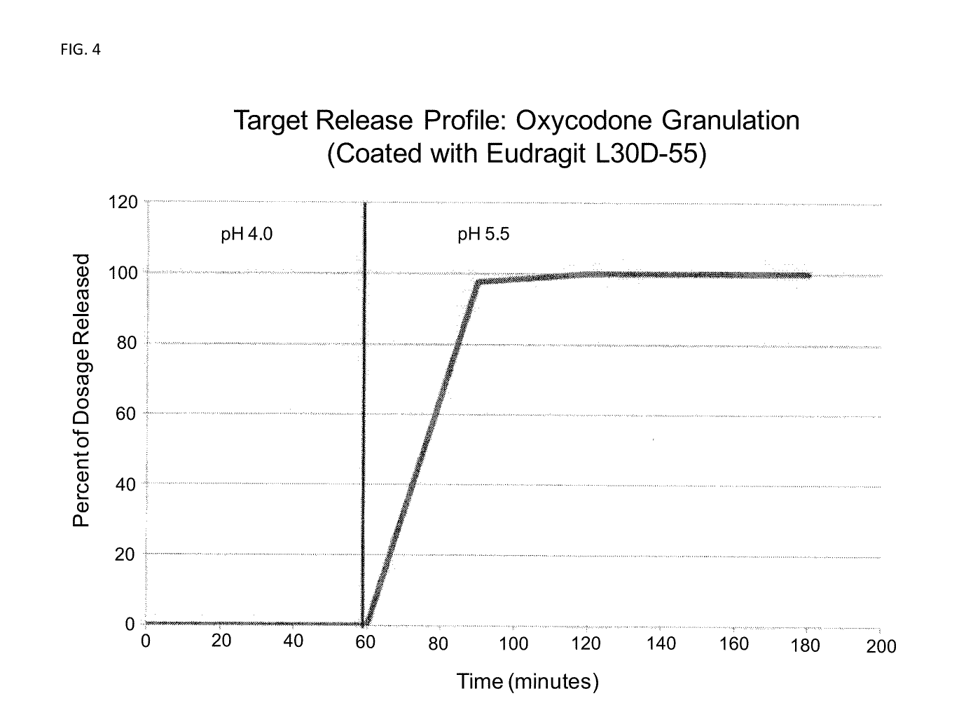

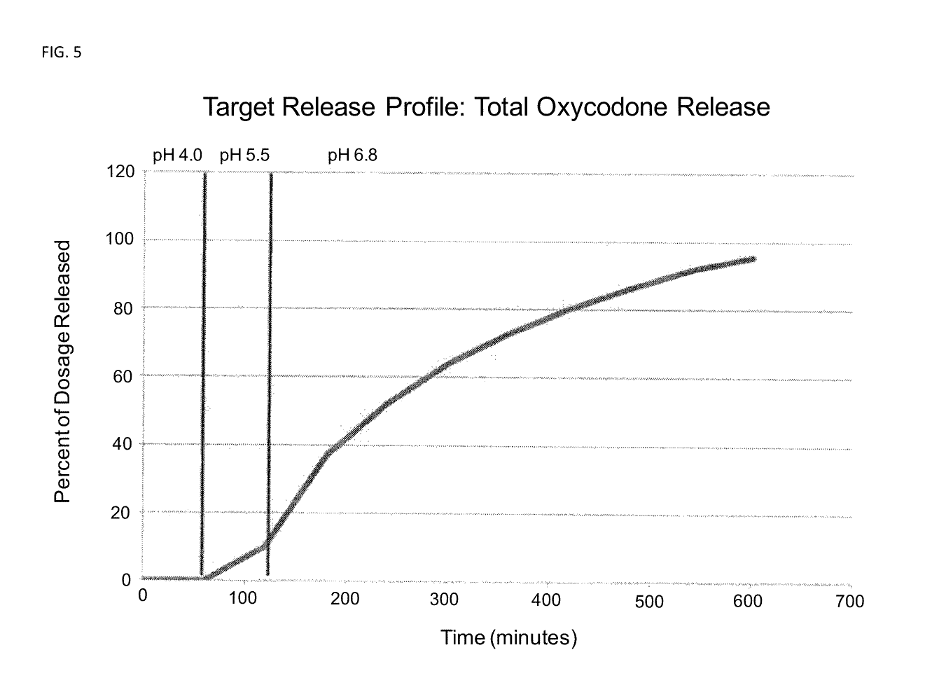

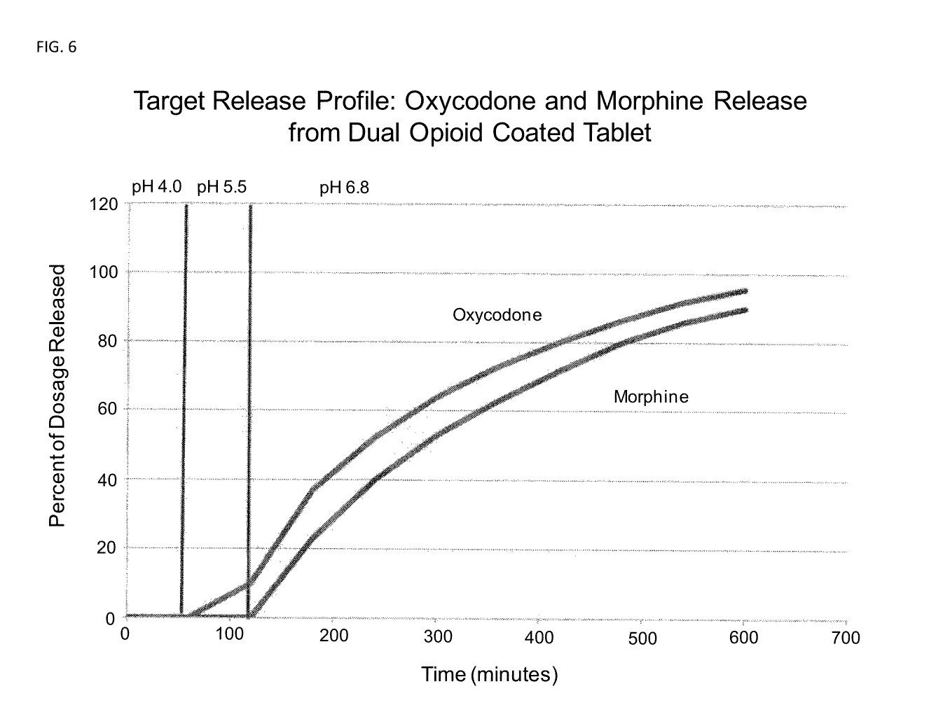

### DETAILED DESCRIPTION OF THE INVENTION

The invention relates to pharmaceutical formulations and methods for the alleviation of acute or chronic pain by controlling the release of compounds having opioid agonist activity for absorption in humans. The pharmaceutical formulations and methods of the invention may provide effective analgesia to a patient while reducing or eliminating undesired side effects typically experienced with the administration of opioid analgesic compounds. Due to the controlled release of the compound (s), it is possible to obtain a substantially constant rate of release of the compound(s) over a specific period of time, corresponding to the dosage necessary for the treatment in question, so that adherence to a strict dosage regimen, e.g. requiring administration of a drug at set intervals up to several times a day, may be dispensed with.

One aspect of the invention relates to pharmaceutical formulations comprising one or more components having one or more release profiles, such that at least one of the components comprises a compound having opioid receptor agonist activity and has a controlled release profile. Another aspect of the invention relates to the administration of the pharmaceutical formulations of the invention to humans in need thereof.

The formulations and methods described herein are used to treat different types of pain, including neuropathic pain and nociceptive pain, somatic pain and visceral pain. In various embodiments, formulations and methods described herein are used to treat diabetic neuropathy, trigeminal neuralgia, postherpetic zoster pain, and thalamic pain syndrome (a central pain). Neuropathic pain frequently coexists with nociceptive pain, and the inventive pharmaceutical formulations and salts may be used to treat mixed pain states, i.e. a combination of neuropathic and nociceptive pain. For example, trauma that damages tissue and nerves, burns (that burn skin as well as nerve endings), and external nerve compression may cause both neuropathic and nociceptive pain. Examples of external nerve compression include tumor nerve compression and sciatica from herniated discs pressing on nerves. In other embodiments, the formulations and methods are used to treat low back pain, cancer pain, osteoarthritis pain, fibromyalgia pain and postoperative pain. In various other embodiments, the formulations and methods are used to treat pain associated with inflammation, bone pain, and joint disease. The formulations and methods of the invention may be used to treat pain caused by a variety of conditions, including, but not limited to, pain after surgery or trauma, pain associated with a medical illness and the like.

The present invention encompasses formulations that can be administered to provide two opioids. An objective of the present invention is to activate certain opioid receptors in the brain by one opioid, and stage the arrival of a second opioid at some timepoint after that receptor is occupied by the first opioid. A dual-opioid extended-release tablet is designed to accomplish this. For example, in formulations that contain oxycodone and morphine, there is a need to delay the release of morphine until the oxycodone is at the receptor by at least one-half hour, and preferably more than one hour. There is also a need to re-supply oxycodone for uptake into the brain at roughly the same rate of elimination from the CNS compartment. It is anticipated that both the delay and the rate of release of oxycodone should approximate one another in the delayed, modified-release pellet components described herein as well as formulations that incorporate the pellets such as, but not limited to, tablets and capsules.

### Compounds Having Opioid Receptor Agonist Activity

The components of the pharmaceutical formulations may comprise a compound having opioid receptor agonist activity. Such compounds may have agonist activity toward the μ-, κ-, α-, or δ-opioid receptors, including other classified receptor subtypes. The compounds having opioid receptor agonist activity may be naturally occurring, semi-synthetic or fully synthetic opiate compounds, derivatives or analogs thereof, or pharmaceutically acceptable salts, esters or prodrugs thereof. Naturally occurring opiates are alkaloid compounds that are found in the resin of the opium poppy, and include morphine, codeine and thebaine. Semi-synthetic or fully synthetic opiates include, but are not limited to, dihydromorphine, heterocodeine, dihydrocodeine, dihydromorphinone, dihydrocodeinone, 3,6-diacetyl morphine, morphinone, 6-desoxymorphine, heroin, oxymorphone, oxycodone, 6-methylene-dihydromorphine, hydrocodone, etorphine, bupemorphine, naloxone or naltrexone.

Compounds having μ-opioid receptor agonist activity may include, but are not limited to, morphine (and structurally related analogs and derivatives), alvimopan, buprenorphine, codeine, 6-desomorphine, dihydromorphine, dihydromorphinone, dihydrocodeine, dihydrocodeinone, 3,6-diacetylmorphine, 6-methylene-dihydromorphine, diphenoxylate, drotebanol, eseroline, etorphine, fentanyl, hydrocodone, levophenacylmorphan, methadone, oxymorphone, nicomorphine, pethidine, picenadol, tapentadole, thebaine, and trimebutane.

Compounds having κ-opioid receptor agonist activity may include, but are not limited to, asimadoline, butorphanol, bremazocine, cyclazocine, dextromethorphan, dynorphin, enadoline, ketazocine, nalbuphine, nalfurafine, norbuprenorphine, oxycodone, pentazocine, salvinorin A, 2-methoxymethyl salvinorin B and its ethoxymethyl and fluoroethoxymethyl homologues, spiradoline, and tifluadom.

Compounds having δ-opioid receptor agonist activity may include, but are not limited to, deltorphin, ethoxymetopon, leu-enkephalin, met-enkephalin, mitragyna speciosa (kratom), mitragynine, mitragynine-pseudoindoxyl, N-phenethyl-14-norbuprenorphine, norclozapine, and 7-spiroindanyloxymorphone.

In certain embodiments, the compound is selected from morphine, codeine, hydromorphone, hydrocodone, oxycodone, dihydrocodeine, dihydromorphine, oxymorphone, mixtures thereof, and pharmaceutically acceptable salts thereof.

Salts include, but are not limited to, hydrochloride, sulfate, bisulfate, tartrate, nitrate, citrate, bitratrate, phosphate, malate, maleate, hydrobromide, hydroiodide, fumarate, succinate and the like.

The components of the pharmaceutical formulations may contain more than one compound, such that the more than one compound is present in a ratio by weight. For example, the components may comprise two compounds, such that the compounds are present in a 2:1, 2:2, 2:3, 2:5, 3:1, or 3:4 weight ratio.

In particular embodiments, the compounds are morphine and oxycodone, or pharmaceutical salts thereof, in ratio of about 3:2 by weight. Pharmaceutical formulations comprising morphine and oxycodone, or pharmaceutical salts thereof, in ratio of about 3:2 by weight, can administer up to a total amount of 18 mg morphine and 12 mg oxycodone per dosage. In some embodiments, pharmaceutical formulations comprising morphine and oxycodone, or pharmaceutical salts thereof, in ratio of about 3:2 by weight, can administer up to an amount of about 600 mg morphine, or pharmaceutical salts thereof, and about 400 mg oxycodone, or pharmaceutical salts thereof, per day. Both oxycodone and morphine exhibit dose propotional/linear pharmacokinetcis. See P. J. Hoskin, et al., The Bioavailability and Pharmacokinetics of Morphine after Intravenous, Oral and Buccal Administration in Healthy Volunteers, Br. J. clin. Pharmac., 27: 499-505, 1989; Robert F. Kaiko, et al., The United States Experience with Oral Controlled-Release Morphine (MS Contin Tablets), Cancer 63:2348-2354, 1989; and Ralph A. Lugo and Steven E. Kern, The Pharmacokinetics of Oxycodone, J. Pain and Palliative Care Pharmacotherapy, 18(4): 17-30, 2004.

### Release Profiles and Characteristics of the Components

At least one of the components in the pharmaceutical formulations comprises a compound having opioid receptor agonist activity and has a controlled release profile.

The formulations may comprise additional components, wherein the additional components may have an immediate release profile or a controlled release profile for the compound.

The term “immediate release” as used herein refers to a release profile in which there is substantially no delay in the release of the compound for absorption.

The term “controlled release” as used herein refers to a release profile in which there is a modification in the release of the compound as compared to an immediate release profile.

Types of controlled release profiles include delayed release, extended release, and pulsatile release profiles.

The term “delayed release” as used herein refers to a release profile in which there is a delay in the release of the compound for absorption. The term “extended release” as used herein refers to a release profile in which the active compound is released at such a rate that blood levels are maintained within the therapeutic range, but below toxic levels, over a period of time of about 8 hours, or about 10 hours, or about 12 hours, or about 15 hours, or about 20 hours, or about 24 hours or about 30 hours, or about 35 hours, or even longer. The term “extended release” differentiates release profile in accordance with the invention from “immediate release” and “delayed release” release profiles. As used herein, “delayed-extended release” refers to release profiles in which release of the active compound is delayed, but is still extended greater than “immediate release” release profiles.

The term “pulsatile release” as used herein refers to a release profile in which the compound is released at intervals for absorption.

### Immediate Release Component

The immediate release component may provide about 1% to about 50% of the total dosage of the compound(s) to be delivered by the pharmaceutical formulation. For example, the immediate release component may provide at least about 5%, or about 10% to about 30%, or about 45% to about 50% of the total dosage of the compound(s) to be delivered by the formulation.

The immediate release component may be a mixture of ingredients that breaks down quickly after administration to release the opioid compound. This can take the form of, for example, granules, particles, powders, liquids and pellets.

### Controlled Release Component

The controlled release component may provide about 30-95% of the total dosage of the compound(s) to be delivered by the pharmaceutical formulation. For example, the controlled release component may provide about 70-90%, or about 80% of the total dosage of the compound(s) to be delivered by the pharmaceutical formulation.

A controlled release component may have a tₘₐₓ of about 1 to about 25 hours following repeated or single administration.

In some embodiments, when the pharmaceutical formulation contains a total dose of about 30 mg of morphine, AUCₜ for morphine is between about 60 ng*h/mL and about 433 ng*h/mL following a single administration of the pharmaceutical formulation. In some embodiments, when the pharmaceutical formulation contains about 30 mg of morphine, Cₘₐₓ for morphine is between about 1 ng/mL and about 11 ng/mL. In certain embodiments, when the pharmaceutical formulation contains about 30 mg of morphine, tₘₐₓ for morphine is between about 3 hours and about 25 hours following a single administration of the pharmaceutical formulation.

In some embodiments, when the pharmaceutical formulation contains a total dose of about 20 mg of oxycodone, AUCₜ for oxycodone is between about 70 ng*h/mL and about 352 ng*h/mL following a single administration of the pharmaceutical formulation. In some embodiments, when the pharmaceutical formulation contains about 20 mg of oxycodone, Cₘₐₓ for oxycodone is between about 5 ng/mL and about 15 ng/mL following a single administration of the pharmaceutical formulation. In certain embodiments, when the pharmaceutical formulation contains about 20 mg of oxycodone, tₘₐₓ is between about 4 hours and about 24 hours following a single administration of the pharmaceutical formulation.

In certain embodiments, plasma concentrations of a drug may be determined after repeated administrations through steady state conditions. As used herein, the term “steady state” means that a plasma level for a given drug has been achieved and which is maintained with subsequent doses of the drug at a level which is at or above the minimum effective therapeutic level and is below the minimum toxic plasma level for compound. For opioid analgesics such as oxycodone, the minimum effective therapeutic level will be partially determined by the amount of pain relief achieved in a given patient. It will be well understood by those skilled in the medical art that pain measurement is highly subjective and great individual variations may occur among patients. It is clear that after the administration of each dose the concentration passes through a maximum and then again drops to a minimum.

The steady state may be described as follows: at the time t=0, the time the first dose is administered, the concentration C is also 0. The concentration then passes through a first maximum and then drops to a first minimum. Before the concentration drops to 0, another dose is administered, so that the second increase in concentration does not start at 0. Building on this first concentration minimum, the curve passes through a second maximum after the second dose has been administered, which is above the first maximum, and drops to a second minimum, which is above the first minimum. Thus, the blood plasma curve escalates due to the repeated doses and the associated step-by-step accumulation of active agent, until it levels off to a point where absorption and elimination are in balance. This state, at which absorption and elimination are in equilibrium and the concentration oscillates constantly between a defined minimum and a defined maximum, is called steady state.

In some embodiments, when the pharmaceutical formulation contains about 30 mg of morphine and the dosing interval is 12 hours, AUC_{ss,τ} is between about 60 ng/mL and about 240 ng/mL. In certain embodiments, when the pharmaceutical formulation contains about 30 mg of morphine and the dosing interval is 12 hours, C_{ss,max} is between about 8 ng/mL and about 29 ng/mL. In some embodiments, when the pharmaceutical formulation contains about 30 mg of morphine and the dosing interval is 12 hours, t_{ss,max} is between about 1 hour and about 5 hours.

In some embodiments, when the pharmaceutical formulation contains about 20 mg of oxycodone and the dosing interval is 12 hours, AUC_{ss,τ} is between about 100 ng/mL and about 550 ng/mL. In certain embodiments, when the pharmaceutical formulation contains about 20 mg of oxycodone and the dosing interval is 12 hours, C_{ss,max} is between about 10 ng/mL and about 50 ng/mL. In some embodiments, when the pharmaceutical formulation contains 20 mg of oxycodone and the dosing interval is 12 hours, t_{ss,max} is between about 1 hour and about 10 hours.

### Active Agents of the Components

The one or more additional components may comprise one or more active agents. For example, the active agents may be any of the compounds having opioid receptor agonist activity as discussed herein.

The active agents may also comprise one or more non-opioid analgesic compound(s), or a mixture of one or more non-opioid analgesic compound(s) and one or more compound(s) with opioid receptor agonist activity, or pharmaceutically acceptable salts, esters or prodrugs thereof. Non-opioid analgesic compounds may act to alleviate pain by other mechanisms not associated with binding to an opioid receptor. For example, the non-opioid analgesic compound may be a non-steroidal anti-inflammatory compound (NSAID), examples of which can include, but are not limited to, piroxicam, lomoxicam, tenoxicam, salicylic acid (aspirin) and other salicylates such as diflunisal; 2-arylpropionic acids such as ibuprofen, carprofen, fenbufen, fenoprofen, flubiprofen, ketoprofen, ketorolac, loxoprofen, naproxen, oxaprozin, tiaprofenic acid and suprofen; n-arylanthranilic acids such as metenamic acid and meclofenamic acid; arylalkanoic acids such as diclofenac, aceclofenac, acemetacin, etodolac, idomethacin, sulindac and tolmetin and the like; or mixtures thereof.

The non-opioid analgesic compound may also be a COX-1 or COX-2 inhibitor compound including, but not limited to, celecoxib (Celebrex®), etoricoxib, lumiracoxib, parecoxib, rofecoxib, valdecoxib, or mixtures thereof. The non-opioid analgesic may also be a calcium channel binding agent such as gabapentin or pregabalin, or a derivative, analog or prodrug thereof, or mixtures thereof.

In certain embodiments, the non-analgesic compound is gabapentin enacarbil (Solzira™), which is a prodrug of gabapentin with the chemical name 1-[[[[1-(2-Methyl-1-oxopropoxy)ethoxy]carbonyl]amino]methyl]cyclohexaneacetic acid. The structures of gabapentin, pregabalin and gabapentin enacarbil are shown below:

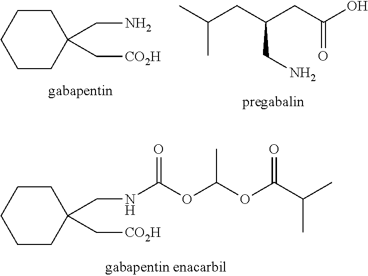 [SMILES: NCC1(CC(=O)O)CCCCC1 ; CC(C)C[C@H](CN)CC(=O)O ; CC(OC(=O)NCC1(CC(=O)O)CCCCC1)OC(=O)C(C)C]

The active agents may further be one or more hybrid opioid compound(s), or a mixture of one or more hybrid opioid compound(s) and one or more compound(s) with opioid receptor agonist activity, or pharmaceutically acceptable salts, esters or prodrugs thereof. Hybrid opioid compounds are compounds formed by covalently binding together two or more opioid compounds with a linker component. The linker component may be stable or may hydrolyze under physiological conditions to provide the parent opioid compounds. Hybrid opioid compounds are described in U.S. Provisional Application Ser. No. 61/153,537 to Holaday et al., filed Feb. 18, 2009. Hybrid opioid compounds are also described in International Patent Application Publication No. WO 2006/073396 to Portoghese et al.

The hybrid opioid compound may comprise two or more compounds having opioid receptor agonist activity, linked by a covalent linker component. The hybrid opioid compound may also comprise a compound having opioid receptor agonist activity linked to a non-opioid active agent including, but not limited to, a non-opioid analgesic compound as described above. In some embodiments, the non-opioid active agent is gabapentin, pregabalin, or gabapentin enacarbil.

The hybrid opioid compound may comprise two or more opiate compounds bonded together by a covalent linker. The opiate compounds may include, but are not limited to, the opiate compounds described above.

The active compounds may be bonded to the linker components by various chemical bonds, preferably at a position on the active agent that does not impair the biological activity of the active agent. Typically, the active agents may be bonded to the linker by a reactive group on the active compound or at a position that may be activated to react with a linker component.

### Preparing the Components

To obtain the components of the pharmaceutical formulations described herein, a combination of excipients is used at appropriate concentrations to provide properties and desired pharmacokinetics. Excipients used in the pharmaceutical formulations described herein are commercially-available, and listed in either the USP or NF. Excipients are selected that will contribute to the function and purpose of each of the active intermediate components and also to the final formulation. One of ordinary skill will appreciate that the concentrations of these excipients used may be increased or decreased as desired to increase or decrease specific properties in a final opioid formulation. Coating materials used herein are also commercially-available and listed in the USP or NF which are incorporated herein by reference.

The technology used to produce a compound-opioid extended-release tablet described herein is a combination of known pharmaceutical manufacturing processes. The unit processes for the manufacture of each active intermediate have been used in several commercially-available products, and therefore are scalable. Two important aspects in producing the compound-opioid extended-release tablet are in the manufacture and performance of the different types of delayed, modified-release pellets. In the example of a dual opioid oxycodone/morphine compound product, the manufacture and performance of the delayed, modified-release oxycodone pellets and the delayed, modified-release morphine pellets is similarly important. These pellets should perform the same as free-flowing, untableted pellets as after tablet compaction. This important feature is best accomplished by adequately plasticizing the coating network to avoid cracking and brittle fracture of the coatings when under compression during tablet compaction.

The materials to be added to the compound(s) for the immediate release component can be, but are not limited to, microcrystalline cellulose, corn starch, pregelatinized starch, potato starch, rice starch, sodium carboxymethyl starch, hydroxypropylcellulose, hydroxypropylmethylcellulose, hydroxyethylcellulose, ethyl-cellulose, chitosan, hydroxychitosan, hydroxymethylated chitosan, cross-linked chitosan, cross-linked hydroxymethyl chitosan, maltodextrin, mannitol, sorbitol, dextrose, maltose, fructose, glucose, levulose, sucrose, polyvinylpyrrolidone (PVP), acrylic acid derivatives (Carbopol, Eudragit, etc.), polyethylene glycols, such a low molecular weight PEGs (PEG2000-10000) and high molecular weight PEGs (Polyox) with molecular weights above 20,000 daltons. It may be useful to have these materials present in the range of 1.0 to 60% (W/W).

In addition, it may be useful to have other ingredients in this system to aid in the dissolution of the drug, or the breakdown of the component after ingestion or administration. These ingredients can be surfactants, such as sodium lauryl sulfate, sodium monoglycerate, sorbitan monooleate, sorbitan monooleate, polyoxyethylene sorbitan monooleate, glyceryl monostearate, glyceryl monooleate, glyceryl monobutyrate, one of the non-ionic surfactants such as the Pluronic line of surfactants, or any other material with surface active properties, or any combination of the above. These materials may be present in the rate of 0.05-15% (W/W).

The materials in controlled release components are the same as the materials in the immediate release component, but with additional polymers integrated into the component, or as coatings over the pellet or granule. The kind of materials useful for this purpose can be, but are not limited to, ethylcellulose, hydroxypropylmethylcellulose, hydroxypropylcellulose, hydroxyethylcellulose, carboxymethylcellulose, methylcellulose, nitrocellulose, Eudragit R, and Eudragit RL, Carbopol, or polyethylene glycols with molecular weights in excess of 8,000 daltons. These materials can be present in concentrations from 4-20% (W/W).

In certain embodiments, components may have pH-sensitive delayed release profiles or non-pH sensitive delayed release profiles. Materials in the pH-sensitive delayed release components may be the same as the materials in the immediate release component, but with additional polymers integrated into the component, or as coatings over the pellet or granule. The kind of materials useful for this purpose can be, but are not limited to, cellulose acetate pthalate, Eudragit L, and other pthalate salts of cellulose derivatives. These materials can be present in concentrations from 4-20% (W/W).

Materials in the pH-sensitive delayed release components may be the same as the materials in the immediate release component, but with additional polymers integrated into the component, or as coatings over the pellet or granule. The kind of materials useful for this purpose can be, but are not limited to, polyethylene glycol (PEG) with molecular weight above 4,000 daltons (Carbowax, Polyox), waxes such as white wax or bees wax, paraffin, acrylic acid derivatives (Eudragit), propylene glycol, and ethylcellulose. Typically these materials can be present in the range of 0.5-25% (W/W) of this component.

### Pharmaceutical Formulations

The pharmaceutical formulations may comprise one or more components having one or more release profiles. Each of the components may comprise the same compound(s), may comprise different compound(s), or a mixture thereof (e.g., some components have the same compounds, other components have different compounds, within the same formulation). In addition, components may comprise active agents as described herein.

For example, the formulations may comprise at least one component, such that the one component has a controlled release profile.

The formulations may also comprise at least two components (a first and second component), such that each components has a different release profile. For example, the second of the at least two components initiates release of the compound(s) contained therein at least one hour after the first component, with the initiation of the release therefrom generally occurring no more than six hours after initiation of release of compound(s) from the first component.

The formulations may also comprise at least three components (a first, second, and third component). The first component may be an immediate release component whereby initiation of release of the compound(s) therefrom is not substantially delayed after administration of the formulation. The second and third components are controlled release components, whereby the release of the compound(s) may be delayed. The controlled release components may be a pH sensitive or a non-pH sensitive delayed component, depending on the type of formulation. The compound(s) released from the delayed release components may be delayed until after initiation of release of the compound(s) from the immediate release component. For example, the compound(s) release from the second component may achieve a Cₘₐₓ at a time after the compound(s) released from the immediate release component may achieve a Cₘₐₓ in the serum. The compound(s) released from the third component may achieve a Cₘₐₓ in the serum after the Cₘₐₓ of the compound(s) released from the second component.

In certain embodiments, the immediate release component may produce a Cₘₐₓ for the compound(s) released therefrom within from about 0.5 to about 2 hours, with the second component producing a Cₘₐₓ for the compound(s) released therefrom in no more than about four hours. In general, the Cₘₐₓ for such a second component may be achieved no earlier than two hours after administration of the formulation; however, it is possible to achieve Cₘₐₓ in a shorter period of time by adjusting the concentration of excipients and/or coatings described herein to achieve a formulation with a desired pharmacokinetic profile.

In certain embodiments, release of compound(s) from the third component may be started after initiation of release of compound(s) from both the first and second components. In some embodiments, Cₘₐₓ for compound(s) released from the third component may be achieved within eight hours.

The formulations may also comprise at least four components (a first, second, third, and fourth component), with each of the at least four components having different release profiles. For example, the compound(s) released from each of the at least four different components may achieve a Cₘₐₓ at a different time.

The formulations may also comprise at least five components (a first, second, third, fourth, and fifth component). The first component may be an immediate release component of a first compound or a first set of compounds, while the second and third components may be controlled release components of the first compound or a first set of compounds. The fourth and fifth components may be controlled release components of a second compound or a second set of compounds. As an example, in certain embodiments, the first compound may be oxycodone and the second compound may be morphine.

In certain embodiments, the formulation may be in the form of a capsule, comprising components that are in the form of separate tablets or pellets. Thus, for example, an immediate release component may be in the form of a tablet or pellet, and controlled release components may be in the form of other tablets or pellets, each of which provides for a delayed release of the compound(s) contained therein, whereby the Cₘₐₓ of the compound(s) released from each of the pellets, or tablets containing the pellets, is reached at different times, with the Cₘₐₓ of the formulation being achieved in less than twelve hours.

In certain embodiments, the pharmaceutical formulation itself will comprise a controlled release profile. In particular embodiments, the pharmaceutical formulation may comprise one or more components that contain two opioid compounds in a 2:1, 2:2, 2:3, 2:5, 3:1, or 3:4 weight ratio. In certain embodiments, the components may comprise morphine and oxycodone in about a 3:2 weight ratio.

As an example, the pharmaceutical formulation may comprise a controlled release component comprising a mixture of morphine and oxycodone, and an immediate release component comprising oxycodone. In some embodiments, the Tₘₐₓ of oxycodone in the immediate release component may be from about 10 minutes to about one hour after ingestion. In other embodiments, the Tₘₐₓ will be from about 10 minutes to about 30 minutes or 45 minutes. The controlled release component may be released at a slower rate and over a longer period of time. For example, in some embodiments, the controlled release component may release effective amounts of the mixture of morphine and oxycodone over 12 hours. In other embodiments, the controlled release component may release effective amounts of morphine and oxycodone over 4 hours or over 8 hours. In still other embodiments, the controlled release component t may release effective amounts of morphine and oxycodone over 15, 18, 24 or 30 hours.

In some embodiments, the later released active agents may be released from the pharmaceutical formulation in pulses so that pulses of the compounds are released at intervals after ingestion of the formulation. For example, in certain embodiments, controlled release component may release a first pulse of the later released active agents about 0.5-1 hour after ingestion, followed by a second pulse after about of 4 hours after ingestion and a third pulse of drug after about 8 hours after ingestion.

### Preparing Formulations

In one aspect, the pharmaceutical formulation may be a solid dosage form, such as a tablet, capsule, soft gelatin capsule, or the like, for oral administration. The pharmaceutical formulation may contain conventional excipients such as binding agents, fillers, lubricants, disintegrants, or wetting agents. In one aspect the tablets or capsules are coated according to methods well known in the art.

The granulation that will best serve this purpose will be highly deformable during compaction, thereby minimizing as much as possible any leakage from the coated pellets before the designated time of release. In one embodiment, it may be desirable to have a brief lag, or delay in the initial burst, or release of oxycodone in the immediate release bolus portion of the formulation. In some embodiments, the tablet is less than about 500 mg, about 450 mg, about 400 mg, about 350 mg, about 300 mg, about 250mg, about 200 mg, about 150 mg, about 100 mg, about 50 mg, about 25 mg, or about 10 mg weight, and the drug load is about 20%, about 15% , about 10% , about 5% (w/w) or less of the formulation. In one embodiment, the goal would be to have as efficient a tablet size as possible, while affording good uniformity and integrity of the pellets in the tablet.

The disintegrant used in the tablet of the present invention is not particularly limited, as far as it is a disintegrant used for pharmaceutical preparations. Examples can include crospovidone, crystalline cellulose, hydroxypropylcellulose with a low degree of substitution, croscarmellose sodium, carmellose calcium, carboxystarch sodium, carboxymethyl starch sodium, potato starch, wheat starch, com starch, rice starch, partly pregelatinized starch, and hydroxypropyl starch. One or two or more of these can be used. Crospovidone is particularly preferable. The sort of disintegrant used for coating granules according to the present invention may be identical to or different from that used inside the granules.

Examples of pharmaceutically acceptable additives used in the tablet of the present invention can include excipients, lubricants, pH adjusters, taste-masking agents, sweeteners, acidifiers, refrigerants, foaming agents, preservatives, fluidizers, antioxidants, colorants, stabilizers, surfactants, buffering agents, flavors, binders and drug solubilizers. A person skilled in the art may immediately list specific examples of these additives.

These additives can be appropriately formulated in the inside of a granule, in the outside of a granule coated with a disintegrant, in the coating of a disintegrant and in all these, as far as they do not damage the advantages of the present invention.

Any lubricant used for pharmaceutical preparation can be used without limitation. Examples of the lubricant used in the tablet of the present invention can include light anhydrous silicic acid, magnesium stearate, stearic acid, calcium stearate, aluminum stearate, aluminum monostearate, sucrose fatty acid esters, polyethylene glycol, sodium stearyl fumarate, stearyl alcohol, talc, titanium oxide, hydrous silicon dioxide, magnesium silicate, synthetic aluminum silicate, calcium hydrogen phosphate, hardened castor oil, hardened rapeseed oil, Carnauba Wax, bees wax, microcrystalline wax and sodium lauryl sulfate. One or two or more kinds of these lubricants can be used. Among these, it is preferable to use one or more selected from light anhydrous silicic acid and magnesium stearate. Particularly, a combination of silicic anhydride contained in the inside of a granule and magnesium stearate contained in the outside of the granule is preferable.

Various hydrophilic polymers may be used with the invention. Examples include, but are not limited to, natural or partially or totally synthetic hydrophilic gums such as acacia, gum tragacanth, locust bean gum, guar gum, and karaya gum; cellulose derivatives such as methyl cellulose, hydroxymethyl cellulose, hydroxypropylmethyl cellulose, hydroxypropyl cellulose, hydroxyethyl cellulose, and carboxymethyl cellulose; proteinaceous substances such as agar, pectin, carrageen, and alginates; hydrophilic polymers such as carboxypolymethylene; gelatin; casein; zein; bentonite; magnesium aluminum silicate; polysaccharides; modified starch derivatives; and other hydrophilic polymers known in the art. An addition example is a carbomer, such as Carbopol 971P.

Diluents increase the bulk of a dosage form and may make the dosage form easier to handle. Exemplary diluents include, but are not limited to, lactose, dextrose, saccharose, cellulose, starch, and calcium phosphate for solid dosage forms, e.g., tablets and capsules; olive oil and ethyl oleate for soft capsules; water and vegetable oil for liquid dosage forms, e.g., suspensions and emulsions. Additional suitable diluents include, but are not limited to, sucrose, dextrates, dextrin, maltodextrin, microcrystalline cellulose (e.g., PH102 or PH200, Avicel®), microfine cellulose, powdered cellulose, pregelatinized starch (e.g., Starch 1500®), calcium phosphate dihydrate, soy polysaccharide (e.g., Emcosoy®), gelatin, silicon dioxide, calcium sulfate, calcium carbonate, magnesium carbonate, magnesium oxide, sorbitol, mannitol, kaolin, polymethacrylates (e.g., Eudragit®), potassium chloride, sodium chloride, and talc.

In embodiments where the pharmaceutical formulation is compacted into a solid dosage form, e.g., a tablet, a binder can help the ingredients hold together. Binders include, but are not limited to, sugars such as sucrose, lactose, and glucose; corn syrup; soy polysaccharide, gelatin; povidone (e.g., Kollidon®, Plasdone®); Pullulan; cellulose derivatives such as microcrystalline cellulose, hydroxypropylmethyl cellulose (e.g., Methocer®), hydroxypropyl cellulose (e.g., Klucel®), ethylcellulose, hydroxyethyl cellulose, carboxymethylcellulose sodium, and methylcellulose; acrylic and methacrylic acid co-polymers; carbomer (e.g., Carbopol®); polyvinylpolypyrrolidine, polyethylene glycol (Carbowax®); pharmaceutical glaze; alginates such as alginic acid and sodium alginate; gums such as acacia, guar gum, and arabic gums; tragacanth; dextrin and maltodextrin; milk derivatives such as whey; starches such as pregelatinized starch and starch paste; hydrogenated vegetable oil; and magnesium aluminum silicate.

When the formulations are in the form of a tablet, the shape of the tablet is not particularly limited, as far as it can be produced without difficulty using an ordinary manufacturing apparatus or a manufacturing apparatus with some modifications. A disc shape that is a general concept for tablets can be mentioned as a typical example. The whole size is not particularly limited. For example, the shorter diameter (diameter for a disc tablet) is appropriately in the range of 6 to 20 mm, preferably 8 to 12 mm. The thickness is neither particularly limited, but appropriately 1 to 10 mm, preferably 2 to 8 mm.

In some embodiments, it may be desirable to have the initial short delay accomplished by adding a delayed-release coating to the tablet which would also serve as a taste-masking agent. This coating may be white, or colored or clear or opaque if desired. An identifying NDC code (in the United States) or similar identifying code may also be printed on the tablet if desired.

The compound used in the tablet of the present invention may be coated with a filmcoating agent, an excipient, a binder, a lubricant, or the like depending on its properties and a plasticizer may be added.

### Anti-Abuse Properties

In another aspect of the invention, the pharmaceutical formulations described herein possess properties that are useful in deterring their use to create formulations that are likely to be used for nonmedical purposes, or as a drug of abuse.

Intentional or inadvertent tampering from extended release formulations will rapidly deliver a massive dose (as a result of converting the sustained release product into an immediate release form) and produce profound a variety of serious and life threatening side effects, including respiratory depression and failure, sedation, cardiovascular collapse, coma and death.

Addicts and recreational drug users commonly use extended release opioids by a variety of routes of administration. Commonly used methods include (a) parenteral (e.g., intravenous injection), (b) intranasal (e.g., snorting), and (c) episodic or repeated oral ingestion of intact or crushed tablets or capsules.

One mode of abuse involves the extraction of the opioid from the component by first mixing the table or capsule with a suitable solvent (e.g., water or alcohol), and then filtering and/or extracting the opioid component from the mixture for intravenous injection. Another mode of abuse of extended release opioids involves dissolving the drug in water, alcohol or another “recreational solvent” to hasten its release and to ingest the contents orally, in order to provide high peak concentrations and maximum euphoriant effects.

The term “tampering” means any manipulation by mechanical, thermal and/or chemical means which changes the physical properties of the component, e.g., to liberate the opioid for immediate release if it is in sustained release form, or to make the opioid agonist available for inappropriate use such as administration by an alternate route, e.g., parenterally. The tampering can be, e.g., by means of crushing, shearing, grinding, mechanical extraction, solvent extraction, solvent immersion, combustion, heating or any combination thereof.

The term “abuse,” “opioid agonist abuse” or “opioid abuse” in the context of the present invention, when it refers to the effects of opioid agonists in causing such, includes intermittent use, recreational use and chronic use of opioid agonists alone or in conjunction with other drugs: (i) in quantities or by methods and routes of administration that do not conform to standard medical practice; (ii) outside the scope of specific instructions for use provided by a qualified medical professional; (iii) outside the supervision of a qualified medical professional; (iv) outside the approved instructions on proper use provided by the drug's legal manufacturer; (v) which is not in specifically approved components for medical use as pharmaceutical agents; (vi) where there is an intense desire for and efforts to procure same; (vii) with evidence of compulsive use; (viii) through acquisition by manipulation of the medical system, including falsification of medical history, symptom intensity, disease severity, patient identity, doctor shopping, prescription forgeries; (ix) where there is impaired control over use; (x) despite harm; (xi) by procurement from non-medical sources; (xii) by others through sale or diversion by the individual into the non-medical supply chain; (xiii) for medically unapproved or unintended mood altering purposes.

The term “abuse resistant,” “abuse deterrent” and “deter abuse” are used interchangeably in the context of the present invention and include pharmaceutical formulations and methods that (i) resist, deter, discourage, diminish, delay and/or frustrate the intentional, unintentional or accidental physical manipulation or tampering of the component (e.g., crushing, shearing, grinding, chewing, dissolving, melting, needle aspiration, inhalation, insufflation, extraction by mechanical, thermal and chemical means, and/or filtration); (ii) resist, deter, discourage, diminish, delay and/or frustrate the intentional, unintentional or accidental use or misuse of the component outside the scope of specific instructions for use provided by a qualified medical professional, outside the supervision of a qualified medical professional and outside the approved instructions on proper use provided by the drug's legal manufacturer (e.g., intravenous use, intranasal use, inhalational use and oral ingestion to provide high peak concentrations); (iii) resist, deter, discourage, diminish, delay and/or frustrate the intentional, unintentional or accidental conversion of an extended release component of the invention into a more immediate release form; (iv) resist, deter, discourage, diminish, delay and/or frustrate the intentional and iatrogenic increase in physical and psychic effects sought by recreational drug users, addicts, and patients with pain who have an addiction disorder; (v) resist, deter, discourage, diminish, delay and/or frustrate the attempts at surreptitious administration of the component to a third party (e.g., in a beverage); (vi) resist, deter, discourage, diminish, delay and/or frustrate attempts to procure the component by manipulation of the medical system and from non-medical sources; (vii) resist, deter, discourage, diminish, delay and/or frustrate the sale or diversion of the component into the non-medical supply chain and for medically unapproved or unintended mood altering purposes; (viii) resist, deter, discourage, diminish, delay and/or frustrate intentional, unintentional or accidental attempts at otherwise changing the physical, pharmaceutical, pharmacological and/or medical properties of the component from what was intended by the manufacturer.

When the component of the pharmaceutical formulation is tampered, the pharmaceutical formulation reduces the amount of opioid agonist released in immediate release form, which in turn reduces the euphoric, pleasurable, reinforcing, rewarding, mood altering and toxic effects of the opioid agonist of the component.

In specific embodiments, the use of certain excipients such as Povidone (Kollidon 30) or Polyoxyl 35 Castor Oil (Cremophor EL™) or Sodium Lauryl Sulfate create an unusable gelatinous mass if tampered with. The addition of aqueous or hydroalcoholic solvents would render the pulverized excipient and drug mixture to a gelatinous mass that would be problematic for easy extraction of the opioid. The Cremophor, in admixture with the methacrylic acid polymers and cellulosic polymers are examples of prime ingredients that cause this feature of the invention.

Other methods of creating abuse-resistant opioid formulations are provided in U.S. published patent application US 20090082466 and U.S. application Ser. Nos. 13/400,004 and 13/400,065, the teachings of which are incorporated herein by reference in their entirety.

For example, abuse-resistant opioid formulation may comprise an abuse deterrent component(s) having a core. The core may comprise a material that has both hydrophilic and hydrophobic properties, such that extraction of the abusable drug by aqueous or alcoholic means is slowed, or even prevented, to an appreciable degree. In certain embodiments, the material may be a viscosity-increasing agent (VIA). Examples of such materials may include, but are not limited to: long-chain carboxylic acids, long-chain carboxylic acid esters, long-chain carboxylic acid alcohols, and/or combinations thereof. The long chain carboxylic acids may generally contain from 6 to 30 carbon atoms and 10 preferably contains at least 12 carbon atoms, most preferably 12 to 22 carbon atoms. In some cases this carbon chain may be fully saturated and unbranched, while others contain one or more double bonds, 3-carbon rings or hydroxyl groups. Examples of saturated straight chain acids are n-dodecanoic acid, n-tetradecanoic acid, n-hexadecanoic acid, caproic acid, caprylic acid, capric acid, lauric acid, myristic acid, palmitic acid, stearic acid, arachidic acid, behenic acid, montanic acid and melissic acid. The long chain carboxylic acids for use in the present invention may also include unsaturated monoolefinic straight chain monocarboxylic acids, which include, but are not limited to oleic acid, gadoleic acid and erucic acid. Also useful are unsaturated (polyolefinic) straight chain monocarboxyic acids. Examples of these are linoleic acid, linolenic acid, arachidonic acid and behenolic acid. Useful branched acids include, for example, diacetyl tartaric acid. Combinations of the straight chain acids are also contemplated.

Examples of long chain carboxylic acid esters include, but are not limited to, those from the group of: glyceryl monostearates; glyceryl monopalmitates; mixtures of glyceryl monostearate and glyceryl monopalmitate (Myvaplex 600, Eastman Fine Chemical Company); glyceryl monolinoleate; glyceryl monooleate; mixtures of glyceryl monopalmitate, glyceryl monostearate, glyceryl monooleate and glyceryl monolinoleate (Myverol 18-92, Eastman Fine Chemical Company); glyceryl monolinolenate; glyceryl monogadoleate; mixtures of glyceryl monopalmitate, glyceryl monostearate, glyceryl monooleate, glyceryl monolinoleate, glyceryl monolinolenate and glyceryl monogadoleate (Myverol 18-99, Eastman Fine Chemical Company); acetylated glycerides such as distilled acetylated monoglycerides (Myvacet 5-07, 7-30 07 and 9-45, Eastman Fine Chemical Company); mixtures of propylene glycol monoesters, distilled monoglycerides, sodium stearoyl lactylate and silicon dioxide (Myvatex TL, Eastman Fine Chemical Company); mixtures of propylene glycol monoesters, distilled monoglycerides, sodium stearoyl lactylate and silicon dioxide (Myvatex TL, Eastman Fine Chemical Company) d-alpha tocopherol polyethylene glycol 1000 succinate (Vitamin E TPGS, Eastman Chemical Company); mixtures of mono- and di-glyceride esters such as Atmul-84 (Humko Chemical 5 Division of Witco Chemical); calcium stearoyl lactylate; ethoxylated mono- and di-glycerides; lactated mono- and diglycerides; lactylate carboxylic acid ester of glycerol and propylene glycol; lactylic esters of long chain carboxylic acids; polyglycerol esters of long chain carboxylic acids, propylene glycol mono- and di-esters of long chain carboxylic acids; sodium stearoyl lactylate; sorbitan monostearate; sorbitan monooleate; other sorbitan esters of long chain carboxylic acids; succinylated monoglycerides; stearyl monoglyceryl citrate; stearyl heptanoate; cetyl esters of waxes; stearyl octanoate; C10-C30 cholesterol/lavosterol esters; and sucrose long chain carboxylic acid esters. Combinations of the long chain carboxylic acid esters are also contemplated.

In certain embodiments, the VIA may be selected from the group consisting of polyacrylic acid, acrylic acid cross-linked with allyl ethers of polyalcohols, hydroxypropyl methylcellulose: hydroxypropyl cellulose mixture, PVP, polyethylene oxide, ethylcellulose, xanthan gum, guar gum, hydroxypropyl cellulose, polyethylene glycol, methacrylic acid copolymer, colloidal silicon dioxide, cellulose gum, starch, sodium starch glycolate, sodium alginate, or combinations thereof. In some embodiments, the VIA may be a carbomers (Carbopol 71G, 971P and 974P), xanthan gum, sodium alginate (Keltone), Polyox, or mixtures thereof.

The materials described above may be co-formulated with a binder, such as, but not limited to, PVP, or its' derivatives, microcrystalline cellulose (Avicel, FMC Corporation), hydroxypropyl methylcellulose, hydroxyethylcellulose, hydroxypropylcellulose, and other cellulose derivatives. In some embodiments, the binder may comprise a hydrophobic oil. Examples of hydrophobic oils include, but are not limited to, a wax, oil, lipid, fatty acids, cholesterol, or triglyceride. In certain embodiments, the binder may be selected from Transcutol, PEG-400 and Cremophor (Castor Oil). Other excipients that may be combined with the VIA include, but are not limited to, lactose, NaHCO3, and magnesium stearate, In certain embodiments, disintegrants or other dispersing agents will not be needed in the abuse deterrent component(s), as the inherent nature of the deconstruction effort in the extraction and abuse of these drug products will cause the materials to be crushed, mixed, and/or disintegrated.

The pellets, beads, beadlets, granules, or the like of the abuse deterrent component(s) may be prepared in multi-stage process that includes (1) blending of the dry powders, (2) wet granulation, (3) extrusion of wet mass, (4) spheronization and (5) drying, as demonstrated in the Examples.

The pellets, beads, beadlets, granules, or the like, of the abuse deterrent component(s) may be coated with an agent that prevents the interaction of the core and the abusable drug. The coating may be pH-sensitive so as not to affect the disintegration process of tablets, or the disaggregation process of capsules or other solid dosage forms within the gut. The coated pellets, beads, beadlets, granules, or the like, may stay largely intact until they pass into the small intestines. To the extent that disintegration of the coated pellets, beads, beadlets, granules, or the like, does occur before the small intestines, it occurs to an unappreciable extent such that the absorption of the active agent is not altered.

In one embodiment, the coating comprises methacrylic acid copolymers (Eudragit L30D-55), hypromellose acetate succinate (AQOAT AS-HF), or a mixture of these two polymer systems. Other pH-sensitive coatings can be, but are not limited to, aqueous acrylic type enteric systems such as Acryl-EZE®, cellulose acetate phthalate, Eudragit L, and other phthalate salts of cellulose derivatives that are pH-sensitive. These materials can be present in concentrations from 4-40% (w/w).

In another embodiment, the coating comprises a functional coating such as a sustained- or controlled-release film coating, or a seal coating and may include Surelease, Opadry® 200, Opadry II, and Opadry Clear.

In another embodiment, the coating comprises plasticizers. An example of a plasticizer is triethyl citrate.

The coated abuse deterrent component(s) may be mixed in any type of solid oral dosage form to make a pharmaceutical formulation of an abusable drug. The abuse deterrent component(s) does not need to be in intimate contact with the abusable drug in order to function in the deterrence of abuse.

### Administration of the Formulation

An aspect of the present invention is a method for treating pain comprising administering a formulation as described herein.

The formulations may be administered, for example, by any of the following routes of administration: sublingual, buccal, transmucosal, transdermal, parenteral, oral etc. In certain embodiments, the formulations may be prepared in a manner suitable for oral administration. Thus, for example, for oral administration, each of the components may be used as a pellet, granule, powder, liquid or a particle, which are then formed into a unitary pharmaceutical product, for example, in a capsule, or embedded in a tablet, or suspended in a liquid for oral administration. The term “formulation” as used herein also refers to a unitary pharmaceutical product containing at least one component.

In certain embodiments, the formulations are for oral administration and may be in the form of a tablet or a capsule or in the form of a multiple unit component. The formulations may be adapted for oral administration 1-6 times a day, normally 1-4 times daily such as 1-3 times, twice daily, or once daily. In the present context the term “once daily” is intended to mean that it is only necessary to administer the pharmaceutical formulation once a day in order to obtain an effective therapeutic amount of the compound to provide a suitable therapeutic response.

The final dose of the compound(s) provided by administration of the formulation may be about, by weight, 100 mg, about 95 mg, about 90 mg, about 85 mg, about 80 mg, about 75 mg, about 70 mg, about 65 mg, about 60 mg, about 55 mg, about 50 mg, about 45 mg, about 40 mg, about 35 mg, about 30 mg, about 25 mg, about 20 mg, about 15 mg, about 12 mg, about 10 mg, about 8 mg, about 5 mg, about 4, mg, about 3 mg, about 2 mg, or about 1 mg.

The dosage of the opioid compound depends on the particular substance, the age, weight condition, etc., of the human or animal that will be treated with the formulation, etc. All such factors are well known to a person skilled in the art.

### EXAMPLES

The present invention will be understood more readily by reference to the following examples, which are provided by way of illustration and are not intended to be limiting of the invention.

### Example 1

### Opioid Components

Components for use in pharmaceutical formulations were developed, as shown in Tables 1-8.

TABLE 1
Target Component 1 (oxycodone):

| | | | |
|---|---|---|---|
| Core: | | | |
| Oxycodone hydrochloride | USP | Drug Substance | 20% |
| Microcrystalline Cellulose | NF | Diluent | 75% |
| (Avicel ® PH-101) | | | |
| Povidone | USP | Binding Agent | 2-4% |
| (Kollidon 30) | | | |
| Polyoxyl 35 Castor Oil | NF | Wetting Agent | 0.5-1.5% |
| (Cremophor EL) | | | |
| Coating: | | | |
| Methacrylic Acid | NF | Functional Film | 12% |
| Copolymer Dispersion | | Sub-Coat | |
| (Eudragit ® L30D-55)¹ | | | |
| Hypromellose Acetate | NF | Functional Film | 48% |
| Succinate (AQOAT | | Over-Coat | |
| AS-HF) | | | |
| Talc | USP | Antitacking Agent | 30% |
| Triethyl Citrate | NF | Plasticizer | 9.5% |
| Sodium Lauryl Sulfate | NF | Wetting Agent | 0.5% |
| Purified Water² | USP | Processing Agent | N/A |

¹Amount per tablet based on the solids content of the dispersion
²Removed during processing

TABLE 2
Target Component 1 (morphine):

| | | | |
|---|---|---|---|
| Core: | | | |
| Morphine Sulfate | USP | Drug Substance | 20% |
| Microcrystalline Cellulose | NF | Diluent | 75% |
| (Avicel ® PH-101) | | | |
| Povidone | USP | Binding Agent | 2-4% |
| (Kollidon 30) | | | |
| Polyoxyl 35 Castor Oil | NF | Wetting Agent | 0.5-1.5% |
| (Cremophor EL) | | | |
| Coating: | | | |
| Methacrylic Acid | NF | Functional Film | 12% |
| Copolymer Dispersion | | Sub-Coat | |
| (Eudragit ® L30D-55)¹ | | | |
| Hypromellose Acetate | NF | Functional Film | 48% |
| Succinate (AQOAT | | Over-Coat | |
| AS-HF) | | | |
| Talc | USP | Antitacking Agent | 30% |
| Triethyl Citrate | NF | Plasticizer | 9.5% |
| Sodium Lauryl Sulfate | NF | Wetting Agent | 0.5% |
| Purified Water² | USP | Processing Agent | N/A |

¹Amount per tablet based on the solids content of the dispersion
²Removed during processing

TABLE 3
Target Component 2 (oxycodone):

| | | | |
|---|---|---|---|
| Core: | | | |
| Oxycodone hydrochloride | USP | Drug Substance | 20% |
| Microcrystalline Cellulose | NF | Diluent | 70-75% |
| (Avicel ® PH-101) | | | |
| Methocel K50 | USP | Binding Agent | 4-9% |
| Polyoxyl 35 Castor Oil | NF | Wetting Agent | 0.5-1.5% |
| (Cremophor EL) | | | |
| Coating: | | | |
| Methacrylic Acid | NF | Functional Film | 12% |
| Copolymer Dispersion | | Sub-Coat | |
| (Eudragit ® L30D-55)¹ | | | |
| Hypromellose Acetate | NF | Functional Film | 48% |
| Succinate (AQOAT | | Over-Coat | |
| AS-HF) | | | |
| Talc | USP | Antitacking Agent | 30% |
| Triethyl Citrate | NF | Plasticizer | 9.5% |
| Sodium Lauryl Sulfate | NF | Wetting Agent | 0.5% |
| Purified Water² | USP | Processing Agent | N/A |

¹Amount per tablet based on the solids content of the dispersion
²Removed during processing

TABLE 4
Target Component 2 (morphine):

| | | | |
|---|---|---|---|
| Core: | | | |
| Morphine Sulfate | USP | Drug Substance | 20% |
| Microcrystalline Cellulose | NF | Diluent | 70-75% |
| (Avicel ® PH-101) | | | |
| Methocel K50 | USP | Binding Agent | 4-9% |
| Polyoxyl 35 Castor Oil | NF | Wetting Agent | 0.5-1.5% |
| (Cremophor EL) | | | |
| Coating: | | | |
| Methacrylic Acid | NF | Functional Film | 12% |
| Copolymer Dispersion | | Sub-Coat | |
| (Eudragit ® L30D-55)¹ | | | |
| Hypromellose Acetate | NF | Functional Film | 48% |
| Succinate (AQOAT | | Over-Coat | |
| AS-HF) | | | |
| Talc | USP | Antitacking Agent | 30% |
| Triethyl Citrate | NF | Plasticizer | 9.5% |
| Sodium Lauryl Sulfate | NF | Wetting Agent | 0.5% |
| Purified Water² | USP | Processing Agent | N/A |

¹Amount per tablet based on the solids content of the dispersion
²Removed during processing

TABLE 5
Target Component 3 (oxycodone):

| | | | |
|---|---|---|---|
| Core: | | | |
| Oxycodone hydrochloride | USP | Drug Substance | 20% |
| Microcrystalline Cellulose | NF | Diluent | 65-75% |
| (Avicel ® PH-101) | | | |
| Methocel E15 | USP | Binding Agent | 4-14% |
| Polyoxyl 35 Castor Oil | NF | Wetting Agent | 0.5-1.5% |
| (Cremophor EL) | | | |
| Coating: | | | |
| Methacrylic Acid | NF | Functional Film | 12% |
| Copolymer Dispersion | | Sub-Coat | |
| (Eudragit ® L30D-55)¹ | | | |
| Hypromellose Acetate | NF | Functional Film | 48% |
| Succinate (AQOAT | | Over-Coat | |
| AS-HF) | | | |
| Talc | USP | Antitacking Agent | 30% |
| Triethyl Citrate | NF | Plasticizer | 9.5% |
| Sodium Lauryl Sulfate | NF | Wetting Agent | 0.5% |
| Purified Water² | USP | Processing Agent | N/A |

¹Amount per tablet based on the solids content of the dispersion
²Removed during processing

TABLE 6
Target Component 3 (morphine):

| | | | |
|---|---|---|---|
| Core: | | | |
| Morphine sulfate | USP | Drug Substance | 20% |
| Microcrystalline Cellulose | NF | Diluent | 65-75% |
| (Avicel ® PH-101) | | | |
| Methocel E15 | USP | Binding Agent | 4-14% |
| Polyoxyl 35 Castor Oil | NF | Wetting Agent | 0.5-1.5% |
| (Cremophor EL) | | | |
| Coating: | | | |
| Methacrylic Acid | NF | Functional Film | 12% |
| Copolymer Dispersion | | Sub-Coat | |
| (Eudragit ® L30D-55)¹ | | | |
| Hypromellose Acetate | NF | Functional Film | 48% |
| Succinate (AQOAT | | Over-Coat | |
| AS-HF) | | | |
| Talc | USP | Antitacking Agent | 30% |
| Triethyl Citrate | NF | Plasticizer | 9.5% |
| Sodium Lauryl Sulfate | NF | Wetting Agent | 0.5% |
| Purified Water² | USP | Processing Agent | N/A |

¹Amount per tablet based on the solids content of the dispersion
²Removed during processing

TABLE 7
Target Component 4 (oxycodone):

| | | | |
|---|---|---|---|
| Core: | | | |
| Oxycodone hydrochloride | USP | Drug Substance | 20% |
| Microcrystalline Cellulose | NF | Diluent | 65-75% |
| (Avicel ® PH-101) | | | |
| Povidone | USP | Binding Agent | 4-14% |
| (Kollidon 25) | | | |
| Polyoxyl 35 Castor Oil | NF | Wetting Agent | 0.5-1.5% |
| (Cremophor EL) | | | |
| Coating: | | | |
| Methacrylic Acid | NF | Functional Film | 12% |
| Copolymer Dispersion | | Sub-Coat | |
| (Eudragit ® L30D-55)¹ | | | |
| Hypromellose Acetate | NF | Functional Film | 48% |
| Succinate (AQOAT | | Over-Coat | |
| AS-HF) | | | |
| Talc | USP | Antitacking Agent | 30% |
| Triethyl Citrate | NF | Plasticizer | 9.5% |
| Sodium Lauryl Sulfate | NF | Wetting Agent | 0.5% |
| Purified Water² | USP | Processing Agent | N/A |

¹Amount per tablet based on the solids content of the dispersion
²Removed during processing

TABLE 8
Target Component 4 (morphine):

| | | | |
|---|---|---|---|
| Core: | | | |
| Morphine sulfate | USP | Drug Substance | 20% |
| Microcrystalline Cellulose | NF | Diluent | 65-75% |
| (Avicel ® PH-101) | | | |
| Povidone | USP | Binding Agent | 4-14% |
| (Kollidon 25) | | | |
| Polyoxyl 35 Castor Oil | NF | Wetting Agent | 0.5-1.5% |
| (Cremophor EL) | | | |
| Coating: | | | |
| Methacrylic Acid | NF | Functional Film | 12% |
| Copolymer Dispersion | | Sub-Coat | |
| (Eudragit ® L30D-55)¹ | | | |
| Hypromellose Acetate | NF | Functional Film | 48% |
| Succinate (AQOAT | | Over-Coat | |
| AS-HF) | | | |
| Talc | USP | Antitacking Agent | 30% |
| Triethyl Citrate | NF | Plasticizer | 9.5% |
| Sodium Lauryl Sulfate | NF | Wetting Agent | 0.5% |
| Purified Water² | USP | Processing Agent | N/A |

¹Amount per tablet based on the solids content of the dispersion
²Removed during processing

### Example 2

### Pharmacokinetic Profile of Opioid Formulations

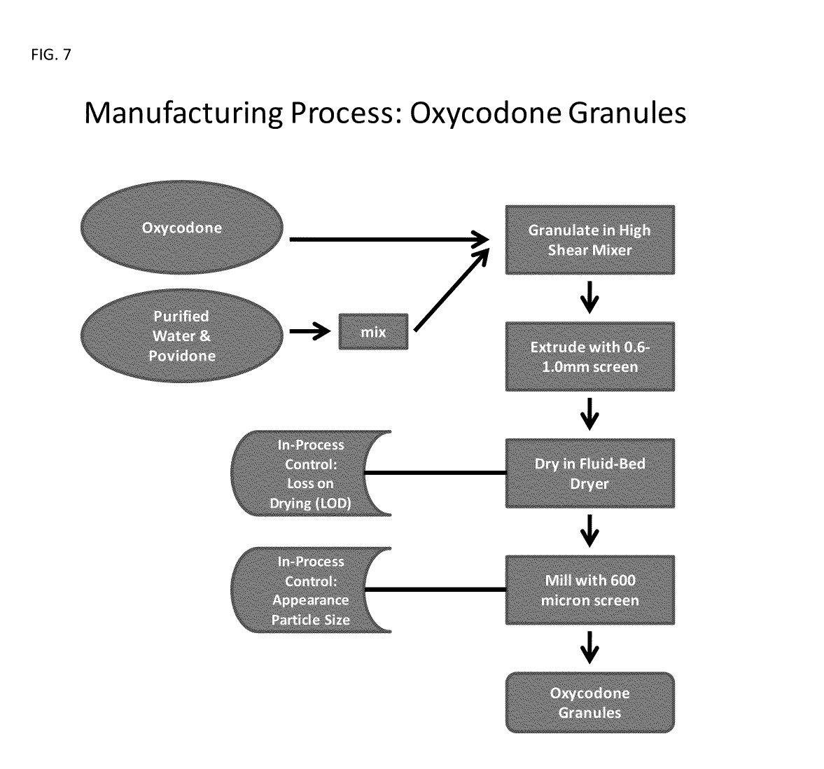

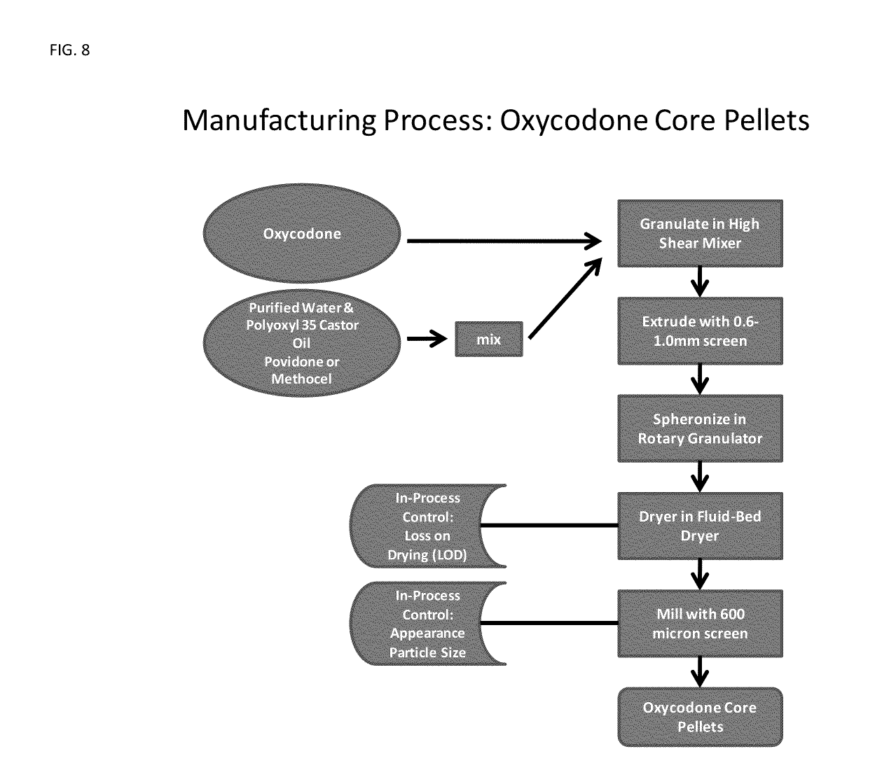

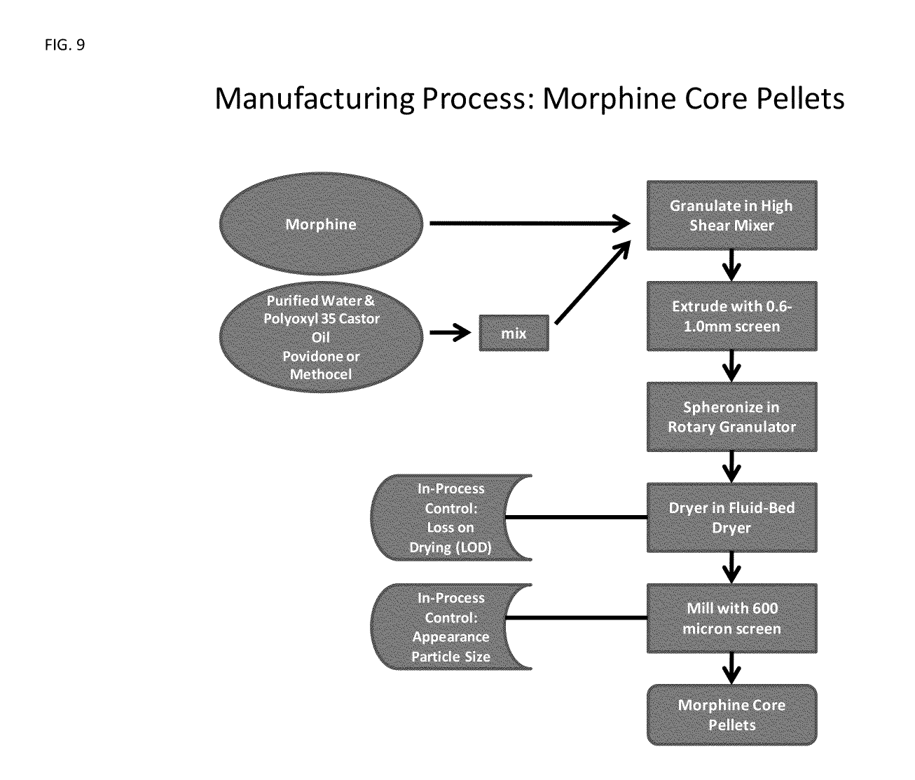

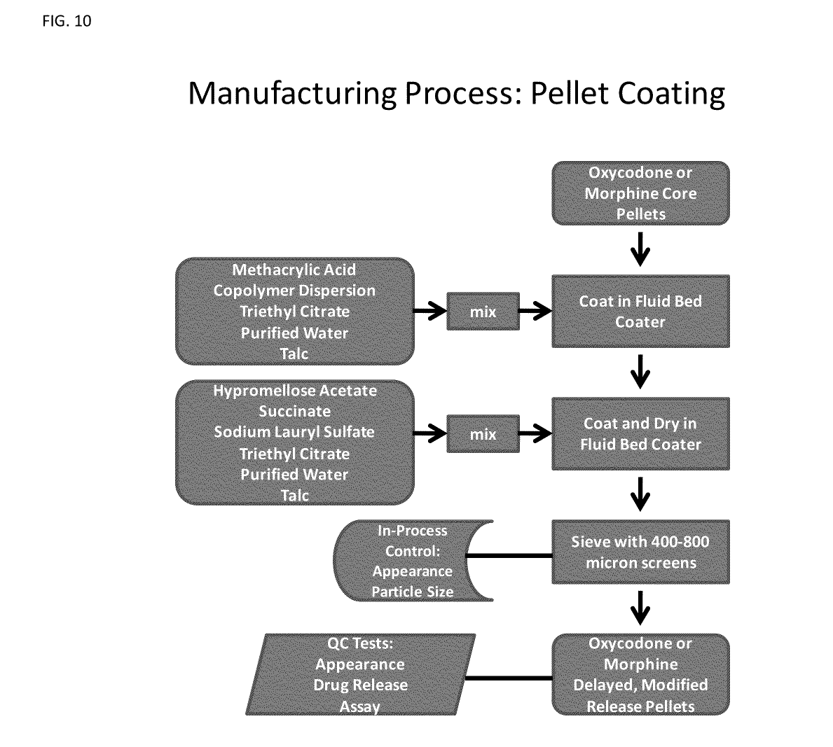

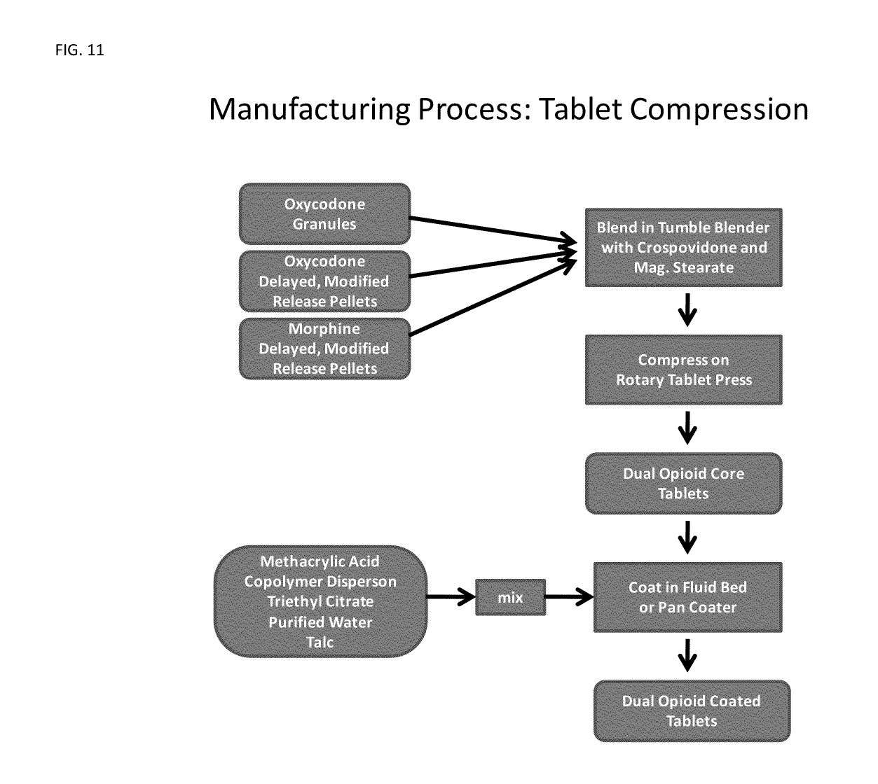

A. An oxycodone formulation is provided that has the following pharmacokinetic profile. The pharmacokinetic profile is achieved by adjusting the concentration of excipients using the methods described in the charts shown in FIGS. 7-11. This 8 mg oxycodone formulation has a Tₘₐₓ of 8 hours and a Tₘᵢₙ of 14 hours.

A controlled release formulation of the instant invention containing 8 mg of oxycodone has a Tₘₐₓ of 8 hours and a Tₘᵢₙ of 14 hours. The pharmacokinetic profile is achieved by adjusting the concentration of excipients using the methods described in the charts shown in FIGS. 7-11.

B. An oxycodone formulation is provided that has the following pharmacokinetic profile. The pharmacokinetic profile is achieved by adjusting the concentration of opioid compound and excipients using the methods described in the charts shown in FIGS. 7-11. This 8 mg oxycodone formulation has a Tₘₐₓ of 6 hours and a Tₘᵢₙ of 16 hours.

C. A dual opioid oxycodone/morphine formulation is provided that has the following pharmacokinetic profile. The pharmacokinetic profile is achieved by adjusting the concentration of opioid compound and excipients using the methods described in the charts shown in FIGS. 7-11. This 8 mg oxycodone/4 mg morphine formulation has a Tₘₐₓ of 6-20 hours for both opioids, and a Tₘᵢₙ of 15-26 hours for both opioids.

D. A dual opioid oxycodone/morphine formulation is provided that has the following pharmacokinetic profile. The pharmacokinetic profile is achieved by adjusting the concentration of opioid compound and excipients using the methods described in the charts shown in FIGS. 7-11. This 18 mg morphine/12 mg oxycodone formulation has a Cₘₐₓ of 6 hours, and a Cₘᵢₙ of 16 hours.

### Example 3

### Preparation of Extended Release Intermediate Formulations

Extended release intermediate formulations A and B were prepared having the components as shown in Tables 9 and 10.

TABLE 9
Formulation A:

| | | | | |
|---|---|---|---|---|
| | Qual- | | | |
| Component | ity* | Function | mg/dose | % w/w |

Core

| | | | | |
|---|---|---|---|---|
| Oxycodone | USP | Drug Substance | 20.00 | 15.19 |
| hydrochloride | | | | |
| Microcrystalline | USP | Filler/Diluent | 75.00 | 56.96 |
| Cellulose | | | | |
| Povidone (Kollidon 30) | USP | Filler/Diluent | 4.00 | 3.04 |
| Polyoxyl 35 Castor Oil | NF | Lubricant | 1.00 | 0.76 |
| Purified Water | USP | Process Aid | — | — |

Barrier Film Coat

| | | | | |
|---|---|---|---|---|
| Ammonio Methacrylate | NF | Film Forming | 1.55 | 1.17 |
| Copolymer, Type A (RL) | | Agent | | |
| Ammonio Methacrylate | NF | Film Forming | 6.18 | 4.70 |
| Copolymer, Type B (RS) | | Agent | | |
| Triethyl Citrate | NF | Plasticizer | 0.77 | 0.59 |
| Magnesium Stearate | NF | Antitacking | 1.50 | 1.14 |
| | | Agent | | |
| Isopropyl Alcohol | USP | Process Aid | — | — |
| Purified Water | USP | Process Aid | — | — |

Enteric Film Coat

| | | | | |
|---|---|---|---|---|
| Methacrylic Acid | NF | Film Forming | 12.75 | 9.68 |
| Copolymer Disp., | | Agent | | |
| Type C | | | | |
| Triethyl Citrate | NF | Plasticizer | 1.28 | 0.97 |
| Talc | NF | Antitacking | 6.38 | 4.84 |
| | | Agent | | |
| Isopropyl Alcohol | USP | Process Aid | — | — |
| Purified Water | USP | Process Aid | — | — |

Dusting Powder

| | | | | |
|---|---|---|---|---|
| Colloidal Silicon | NF | Lubricant | 1.26 | 0.96 |
| Dioxide | | | | |
| Total | | | 131.66 | 100.00 |

*USP = United States Pharmacopeia; NF = National Formulary

TABLE 10
Formulation B:

| | | | | |
|---|---|---|---|---|
| | Qual- | | | |
| Component | ity* | Function | mg/dose | % w/w |

Core

| | | | | |
|---|---|---|---|---|
| Oxycodone | USP | Drug | 20.00 | 13.92 |
| hydrochloride | | Substance | | |
| Microcrystalline | USP | Filler/Diluent | 75.00 | 52.22 |
| Cellulose | | | | |
| Povidone (Kollidon 30) | USP | Filler/Diluent | 4.00 | 2.79 |
| Polyoxyl 35 Castor Oil | NF | Lubricant | 1.00 | 0.70 |
| Purified Water | USP | Process Aid | — | — |

Barrier Film Coat

| | | | | |
|---|---|---|---|---|
| Ammonio Methacrylate | NF | Film Forming | 3.09 | 2.15 |
| Copolymer, Type A (RL) | | Agent | | |
| Ammonio Methacrylate | NF | Film Forming | 12.36 | 8.61 |
| Copolymer, Type B (RS) | | Agent | | |
| Triethyl Citrate | NF | Plasticizer | 1.55 | 1.08 |
| Magnesium Stearate | NF | Antitacking | 3.00 | 2.09 |
| | | Agent | | |
| Isopropyl Alcohol | USP | Process Aid | — | — |
| Purified Water | USP | Process Aid | — | — |

Enteric Film Coat

| | | | | |
|---|---|---|---|---|
| Methacrylic Acid | NF | Film Forming | 13.91 | 9.68 |
| Copolymer Disp., | | Agent | | |
| Type C | | | | |
| Triethyl Citrate | NF | Plasticizer | 1.39 | 0.97 |
| Talc | NF | Antitacking | 6.95 | 4.84 |
| | | Agent | | |
| Isopropyl Alcohol | USP | Process Aid | — | — |
| Purified Water | USP | Process Aid | — | — |

Dusting Powder

| | | | | |
|---|---|---|---|---|
| Colloidal Silicon | NF | Lubricant | 1.38 | 0.96 |
| Dioxide | | | | |
| Total | | | 143.63 | 100.00 |

*USP = United States Pharmacopeia; NF = National Formulary

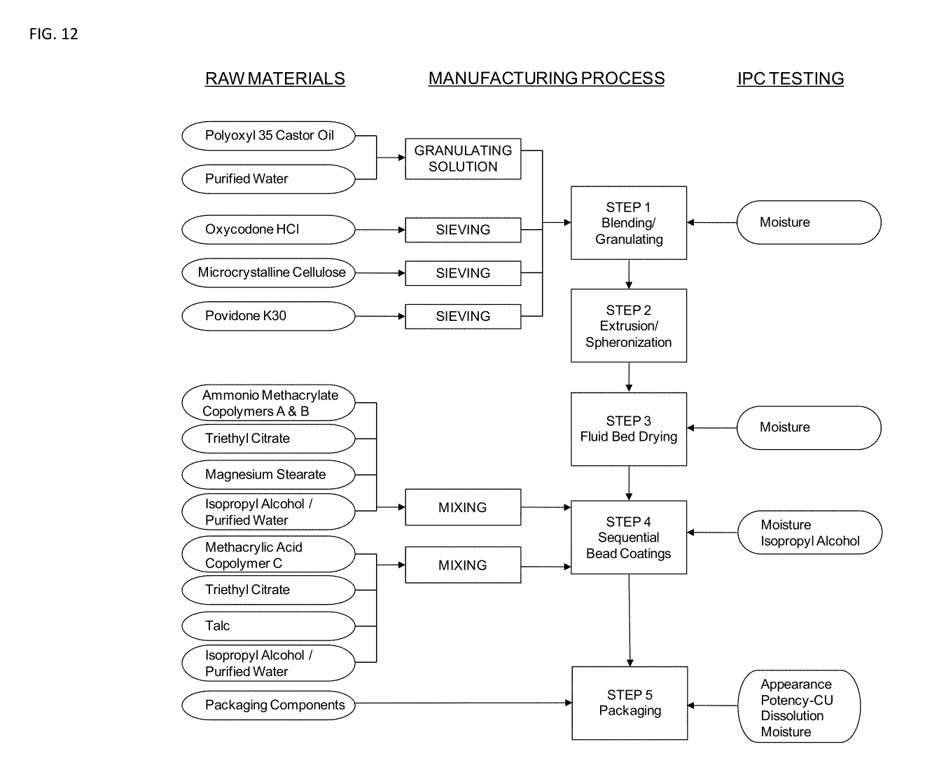

The manufacturing process of mixing the formulations is illustrated in the flow diagram of FIG. 12. To prepare the formulations, oxycodone hydrochloride, microcrystalline cellulose, and Povidone (Kollidon 30) were individually, manually screened through a #20 mesh screen into a collecting container. The screened mix was transferred to a granulation bowl of a high shear granulator and dry mixed for three minutes.

A granulating solution comprising purified water mixed with Polyoxyl 35 Castor Oil was sprayed at a constant rate into the granulation bowl, mixing at low-speed-impeller or low-speed-chopper setting. The resulting granulation mixture was visually assessed continuously, and additional purified water was sprayed onto the mass as required.

The granulation mixture then underwent an extrusion-spheronization process using an extruder and plate spheronizer. The wet mass was uniformly extruded through a 0.8 mm screen into the marmurizing bowl where the extrudate was formed into appropriate sized pellets.

The pellets were dried using a Fluid Bed Dryer Granulator to a Loss on Drying (LOD) test target of ≦3%. To obtain the preferred fraction, the dried pellets were sieved through a #20 and #40 mesh size stainless steel screen into a double polyethylene-lined fiber drum for storage pending pellet spray coating.

The pellets then underwent spray coating using a Fluid Bed Dryer. In a stainless steel vessel, the coating components were mixed into an isopropyl alcohol/water solution using a pneumatic propeller mixer for at least one hour until a clear solution resulted. In a separate stainless steel vessel, the enteric coating solution was prepared by mixing the enteric coating components with a pneumatic mixer for at least one hour until a clear solution resulted. The polymer coating solutions were sprayed onto the pellets while continuously monitoring the spray conditions. The completed pellets were discharged into a double polyethylene-lined fiber drum for work-in-process storage pending lubrication.

The lubricated pellets were sieved through a #18 and #40 mesh size stainless steel screen to obtain the preferred fraction, and discharged into a double polyethylene-lined fiber drum for storage pending tablet blending.

### Example 4

### Pharmacokinetic Testing of Formulations A and B

Methods

A single-dose, three-period, three-sequence, three-treatment crossover study was conducted to compare the oxycodone pharmacokinetic profile in human subjects following oral administration of Formulation A or B as described in Example 3, or with a Reference Formulation (MS Contin® 30 mg (morphine CR) co-administered with OxyContin® 20 mg (oxycodone CR)).

Each subject participated in a series of three periods, wherein each period was comprised of (i) pre-administration screening and check-in, (ii) administration of the formulation, and (iii) post-administration sample collection and follow-up. The subjects received a different formulation in each period, and were divided randomly to determine in which order the formulations were administered.

The pre-administration screening and check-in involved a physical examination and recordation of the subject's vital signs. Naltrexone (50 mg), an opioid antagonist, was administered 0.5 hours prior to administration. Blood samples were collected at 10 minutes and after 0.5, 1, 2, 3, 4, 5, 5.5, 6, 6.5, 7, 8, 10, 12, 14, 18, 21, 24, 48, and 72 hours post-dose of the formulation.

Morphine and oxycodone in the plasma of the blood samples were measured by liquid chromatography with tandem mass spectrometry (LC/MS/MS) methods that were validated across the following ranges:

Morphine 0.25-100 ng/mL

Oxycodone 50-50,000 pg/mL

Results

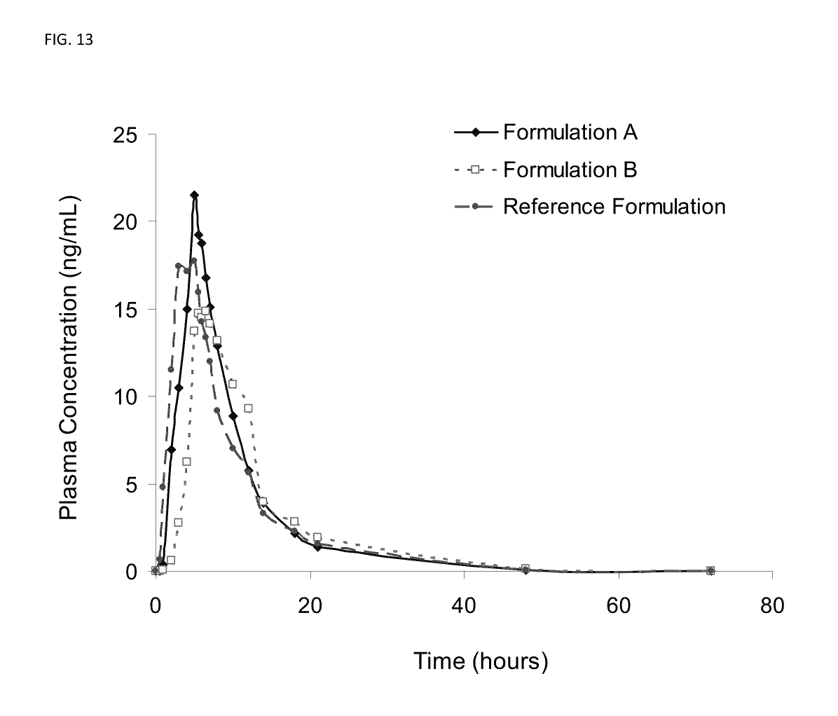

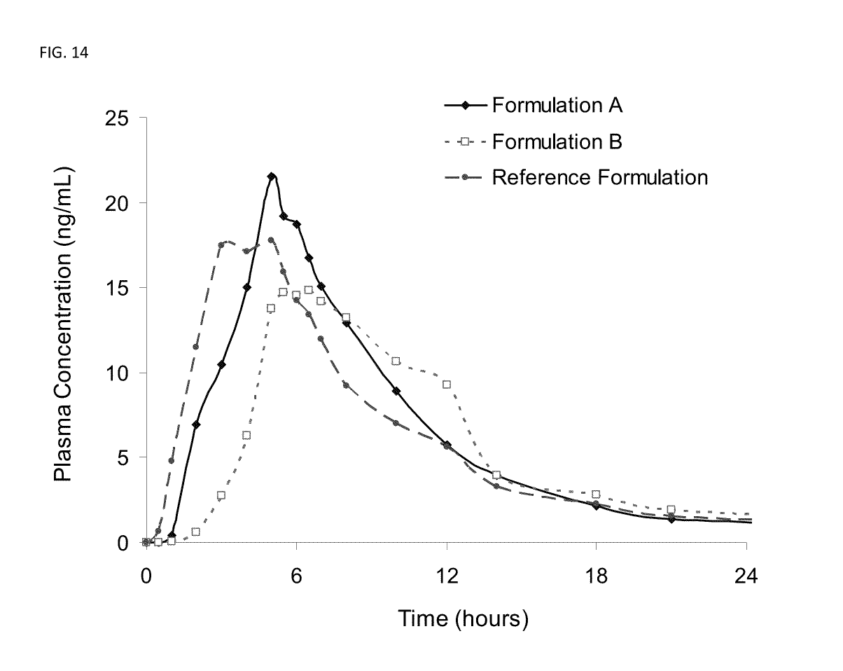

The mean plasma concentration of oxycodone at the sample collection timepoints is shown in FIG. 13 (through 72 hours) and FIG. 14 (the first 24 hours). As compared to the Reference Formulation, Formulation A resulted in higher plasma levels of oxycodone between 5 and 16 hours after treatment, although the plasma levels were generally lower thereafter. On other hand, Formulation B produced about the same or greater plasma levels of oxycodone as compared to the Reference Formulation at 6 hours after treatment and continuing through 48 hours. During this period, the plasma levels of oxycodone provided by Formulation B were, on average, 30% greater than the plasma levels provided by the Reference Formulation.

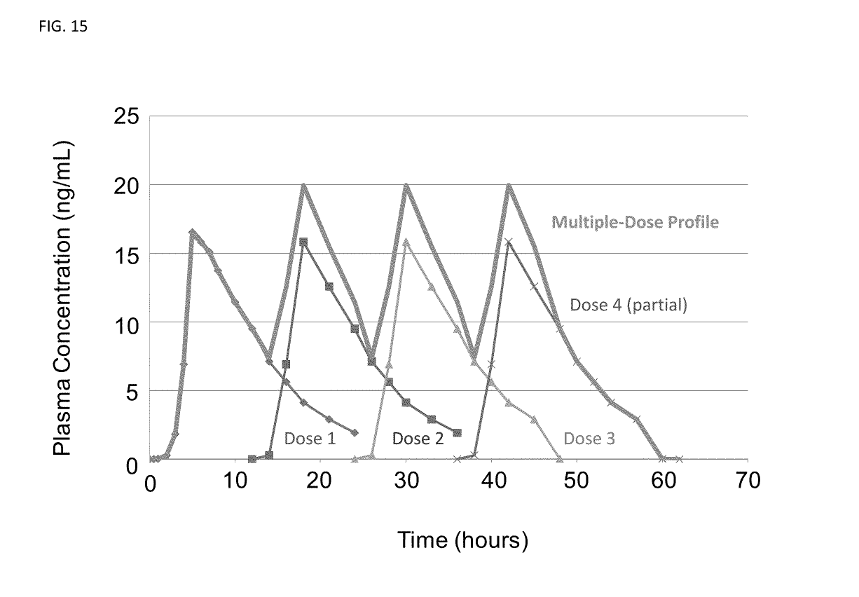

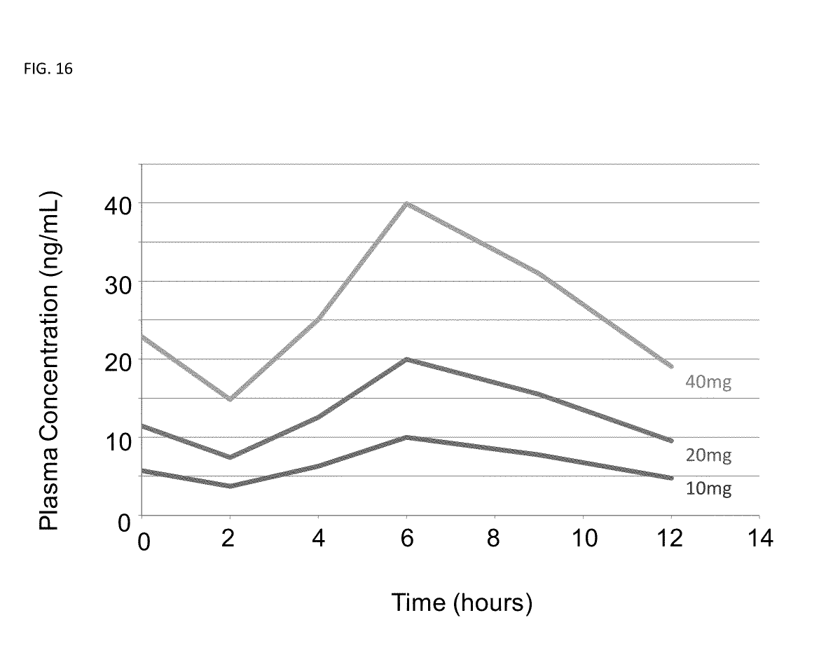

These data were used to project oxycodone plasma profiles that would result from administering multiple doses of Formulation B, as shown in FIGS. 15 and 16. FIG. 15 presents the oxycodone plasma profile through administration of 4 doses of Formulation B and indicates that, under this dosing regimen, oxycodone plasma levels can be maintained between about 7 and about 20 ng/mL.

FIG. 16 shows the oxycodone plasma profile that may result from different dosing strengths, and focuses on a single dose with the multiple dose regimen after the plasma levels achieve a steady-state; steady state is characterized by consistent peaks and troughs in the multiple dose plasma profile. FIG. 16 indicates that, at steady state, Cₘₐₓ will reflect the strength of the administered dose.

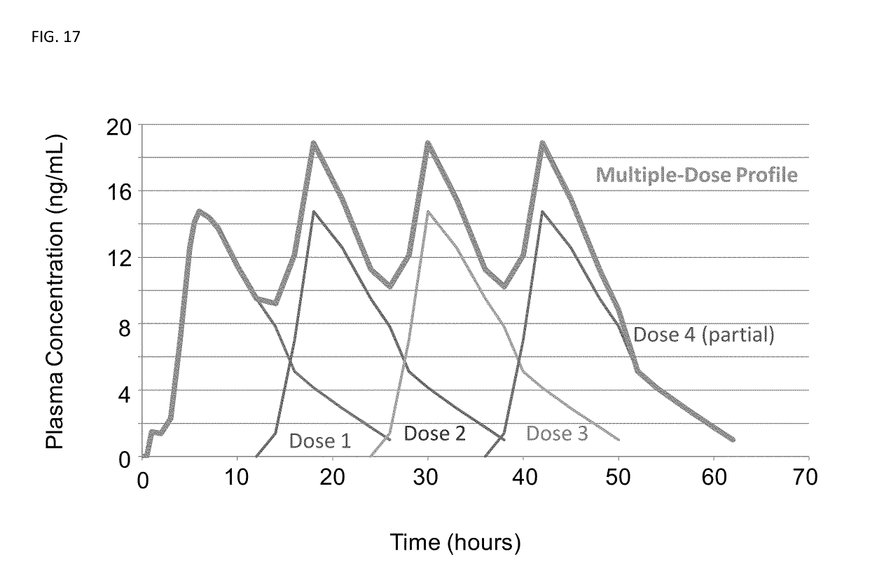

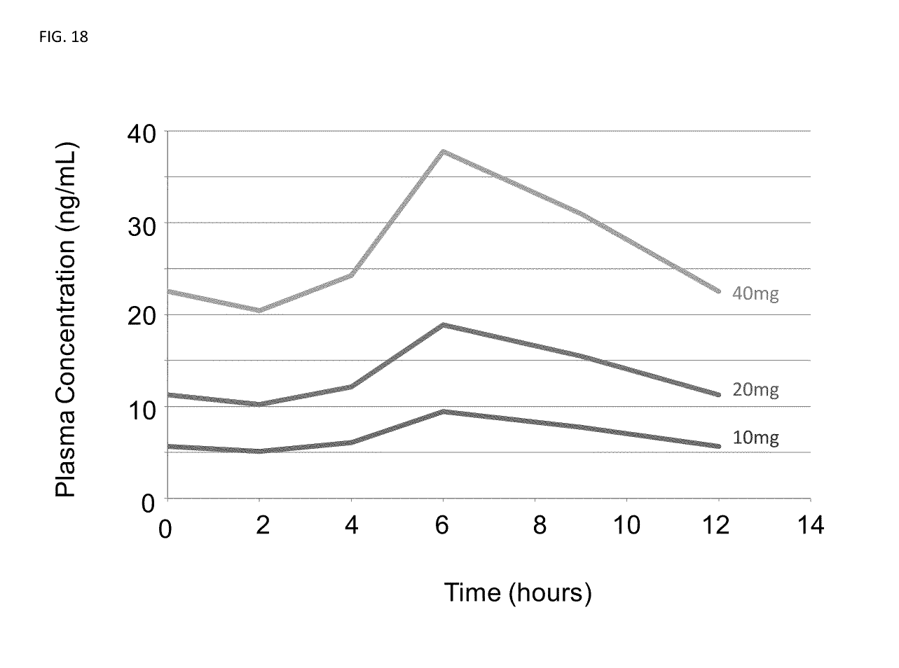

FIGS. 17 and 18 present projections of the oxycodone plasma profile for multiple doses of a formulation comprising a composite of an immediate release formulation (10%) and Formulation B (90%). FIG. 17 demonstrates the oxycodone plasma profile through administration of 4 doses of the composite formulation and indicates that, under this dosing regimen, oxycodone plasma levels can be maintained between about 10 and about 19 ng/mL.

FIG. 18 shows the oxycodone plasma profile that may result following administration of the composite formulation at different dosing strengths. FIG. 18 focuses on a single dose with the multiple dose regimen after the plasma levels achieve a steady-state, which is characterized by consistent peaks and troughs in the multiple dose plasma profile. The projection indicates that, at steady state, Cₘₐₓ will be less than the administered dose.

Comparisons of the oxycodone plasma profile of Formulation A to the Reference Formulation and the oxycodone plasma profile of Formulation B to the Reference Formulation are shown in Tables 11 and 12.

TABLE 11
Comparing Oxycodone Pharmacokinetics of Formulation
A and the Reference Formulation

| | Formulation A | Reference Formulation |
|---|---|---|
| AUCₜ [pg · hr/mL] | 167077.87 ± 18761.51 | 194706.30 ± 41996.62 |
| Cₘₐₓ [pg/mL] | 24410.50 ± 4864.72 | 20525.70 ± 4520.50 |
| Tₘₐₓ [h] | 5.00 (2.00-6.00) | 3.00 (2.00-5.00) |

TABLE 12
Comparing Oxycodone Pharmacokinetcis of Formulation
B and the Reference Formulation

| | Formulation B | Reference Formulation |
|---|---|---|
| AUCₜ [pg · hr/mL] | 180846.58 ± 22868.36 | 194706.30 ± 41996.62 |
| Cₘₐₓ [pg/mL] | 16471.00 ± 3543.53 | 20525.70 ± 4520.50 |
| Tₘₐₓ [h] | 5.75 (5.00-12.0) | 3.00 (2.00-5.00) |

While AUCₜ of Formulations A and B were less than AUCₜ of the Reference Formulation, AUCₜ of Formulations A and B were within 14% and 7%, respectively. Also, Tₘₐₓ of both Formulations A and B were greater than Tₘₐₓ of the Reference Formulation, which was not expected.

### Example 5

### Immediate-Release Formulation of Oxycodone with Controlled Release Mixture of Oxycodone-Morphine

A solid oral component tablet, comprising a core of 5.0 mg oxycodone hydrochloride and 5.0 mg morphine sulfate as active ingredients together with ammonio methacrylate copolymer, hypromellose, lactose, magnesium stereate, polyethylene glycol 400, povidone, sodium hydroxide, sorbic acid, stearyl alcohol, talc, titanium dioxide and triacetin, is prepared according to standard methods known in the art for preparation of tablets. The outside of the tablet is coated with a controlled release formulation comprising 10 mg of oxycodone hydrochloride and gelatin, hypromellose, maize starch, polyethylene glycol, polysorbate 80, red iron oxide, silicon dioxide, dodium laurel sulfate, sucrose, titanium dioxide and yellow iron oxide. The resulting tablet is administered to patients for the alleviation of pain and results in effective analgesia with no incidence of morphine-induced respiratory depression.

### Example 6

### General Procedure for Preparation of Controlled Release Formulations

The following manufacturing description is provided by way of example for the preparation of a controlled release, compressed tablet containing morphine sulfate and oxycodone hydrochloride.

### Preparation of Cores

The active drug substances (morphine sulfate and oxycodone hydrochloride), microcrystalline cellulose, USP and Povidone K30, NF were individually manually screened through a #20 mesh screen into a collecting container. The screened mix was transferred to the granulation bowl of a high shear granulator such as the PMA-25 or PMA-65 and dry mixed for 3 minutes.

A granulating solution consisting of a previously mixed solution of Purified Water, USP and Polyoxyl 35 Castor Oil, NF was sprayed at a constant rate into the granulation bowl and mixed at low speed impeller/low speed chopper setting. Granulation outcome was visually assessed on a continuous basis and additional Purified Water, USP was sprayed onto the mass if required. At the end of the granulation period, a sample was removed for an in-process test for water content.

After sampling was completed, the granulation was discharged to the extrusion-spheronization process using a Luwa extruder and plate spheronizer or equivalent. The wet mass was uniformly extruded through a 0.8 mm screen into the marmurizing bowl where the extrudate was formed into appropriate sized pellets.

Fluid bed drying of the pellets was conducted using suitable process parameters with a GPCG-3, GPCG-5 or equivalent to a Loss on Drying (LOD) test target of ≦5%. The dried pellets were sieved to obtain the preferred fraction through a #20 and #40 mesh size stainless steel screen into a double PE-lined fiber drum for work-in-process storage pending pellet spray coating.

### Preparation of Modified Release Coated Beadlets

The ammonio methacrylate copolymers and triethyl citrate were mixed using a pneumatic propeller mixer into an isopropyl alcohol/water solution contained in a stainless steel vessel for at least one hour until a clear solution was obtained. Talc was then added to the vessel with continuous stirring. Fluid bed spray coating of the core pellets was conducted using suitable process parameters with a GPCG-5 Wurster fitted with a 1.0 mm spray nozzle.

### Preparation of Enteric Coated Beadlets

In a separate container, the enteric coating solution was prepared by mixing methacrylic acid copolymer and triethyl citrate with a pneumatic mixer in a stainless steel vessel for at least one hour. Talc was then added to the vessel with continuous stirring. The polymer coating solutions were successively sprayed at a constant rate to completion onto the beadlets while the spray conditions were continuously monitored. The enteric coated beadlets were discharged into a double polyethylene-lined fiber drum for work-in-process storage pending lubrication.

### Example 7

### Dissolution Testing Method for Controlled Release Formulations

The dissolution test method was designed to be used with an automated dissolution sampling station (e.g., Varian VK 8000). If such an instrument is not available, appropriate adjustments can be made in order to pull samples manually.
Apparatus: USP <711> Apparatus 2 (Paddles) Automated Dissolution Sampling Station
Vessel Size/Type: About 1000 mL/clear glass, round-bottom vessel
Rotation Speed: About 50 rpm throughout
Media and Volume: Stage 1 (Acid Stage) from 0-2 hours: 750 mL of acidic Dissolution Medium A at 37.0±0.5° C. for 2 hours
   Stage 2 (Buffer Stage) from 2-11 hours: 1000 mL at 37.0±0.5° C., created by adding 250 mL of Dissolution Medium B and 20 mL Dissolution Medium A to the remnants of the media in the vessel from Stage 1. The Stage 2 media should have a pH of about 6.8
Test Temperature: About 37.0±0.5° C.
Sinker: Basket Sinker (0.46″×0.80″) 40 Mesh, 316-SS wire cloth
Pull Volume: About 10 mL
Profile Time-points: About 1, 2, 3, 4, 6, 9 and 11 hours
Media Replacement: No
Sampling: Automated
Filter Type/Size: In-line 10-μm polyethylene full flow filter

### Example 8

### Controlled Release Opioid Formulation Formulations

Following the procedure of Example 6, the following formulations were prepared:

TABLE 13
Modified Release Beadlets Formulations Using Ammonio
Methacrylate Copolymer Having RS/RL Ratio of 90/10.

| | Quantity per | % per |
|---|---|---|
| Component | Unit (mg) | Unit (w/w) |
| Morphine Sulfate + Oxycodone HCl | 40 | 62.5 |
| Core Pellets | | |
| Ammonio Methacrylate copolymer | 16.3 | 25.5 |
| Type B (RS PO) | | |
| Ammonio Methacrylate copolymer | 1.8 | 2.8 |
| Type A (RL PO) | | |
| Triethyl Citrate NF/EP | 2.3 | 3.5 |
| Talc (197 Grade) USP/EP/JP | 3.6 | 5.6 |

| | |
|---|---|
| Water purified | Removed by evaporation |
| | during the coating process |

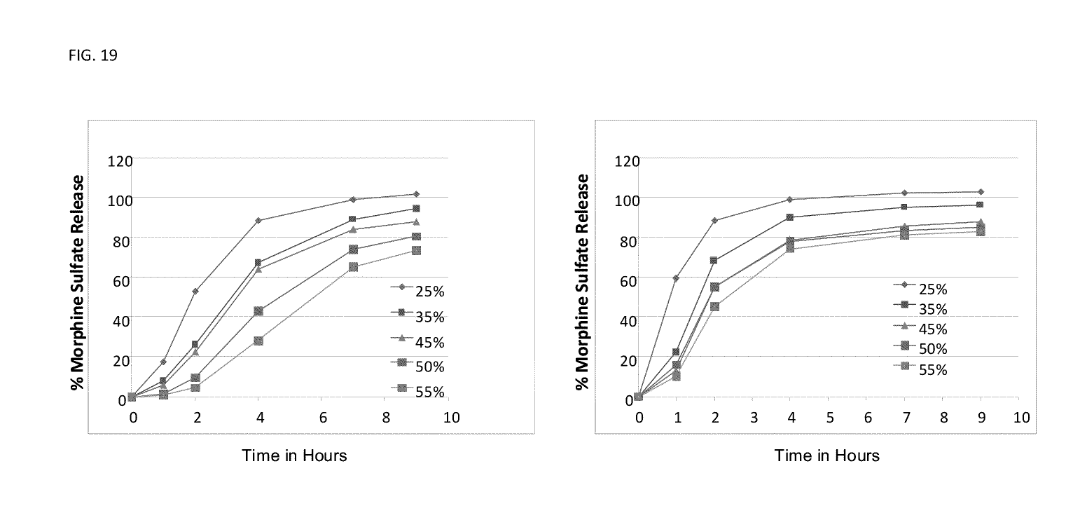

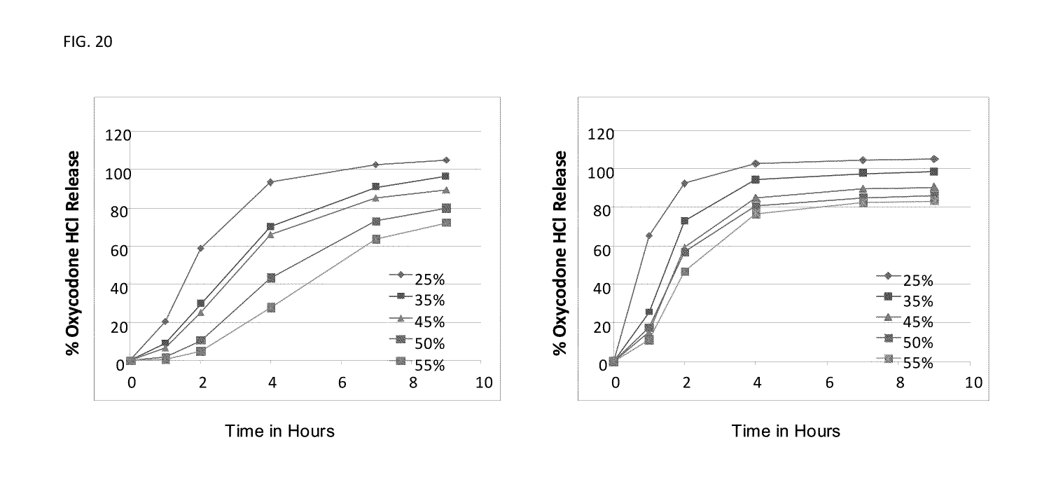

Various formulations were prepared having different % coating levels (e.g., 25%, 35%, 45%, 50% and 55%) of the ammonio methacrylate RS/RL polymers. FIGS. 19(a) and 20(a) provide representative dissolution profiles for morphine sulfate and oxycodone hydrochloride, respectively.

TABLE 14
Tablet Formulations Using Morphine/Oxycodone
Enteric Coated/Modified Release Beadlets.

| | Quantity per | % per |
|---|---|---|
| Component | Unit (mg) | Unit (w/w) |
| Morphine Sulfate + Oxycodone HCl | 20 | 20 |
| Modified Release Beadlets (RS/RL = | | |
| 90:10 + Enteric Coating) | | |
| Microcrystalline Cellulose PH101 | 73.1 | 73.1 |
| Granulated | | |
| Povidone K30 | 6.4 | 6.4 |
| Magnesium Stearate 5712 | 0.5 | 0.5 |

| | |
|---|---|
| 8% w/w Povidone solution | Water removed by |
| (used for granulation) | evaporation post process. |

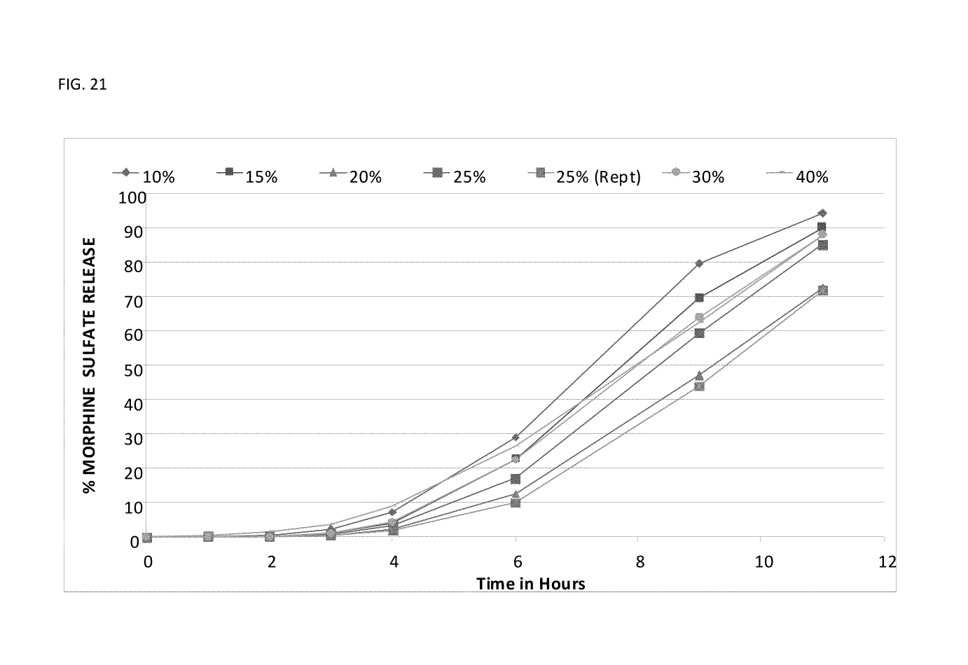

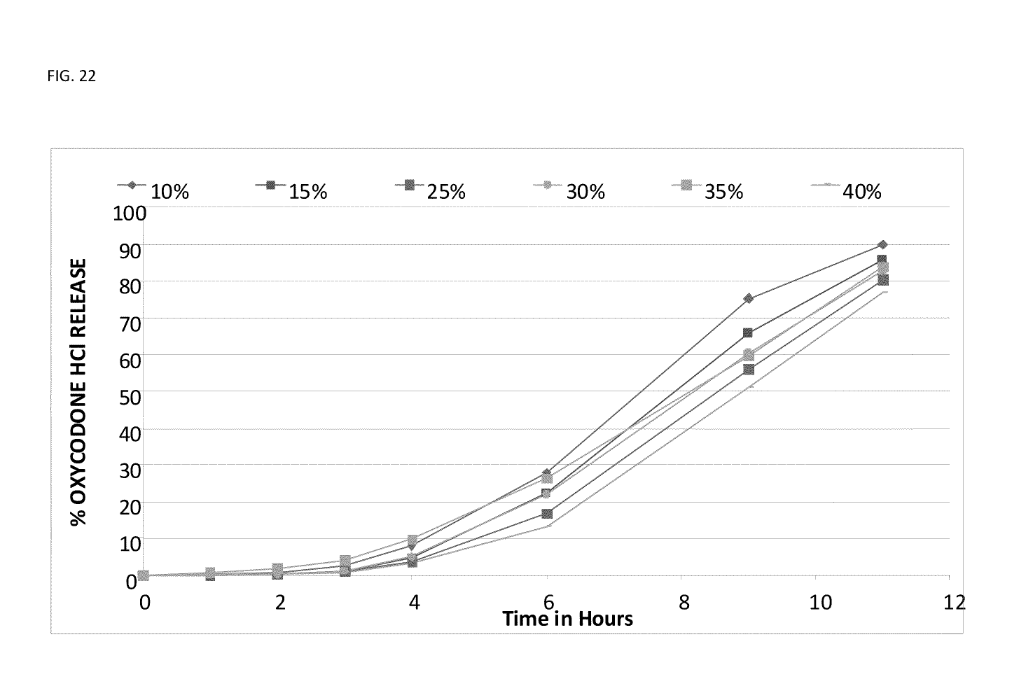

Various tablet formulations were prepared having different % enteric coating levels (e.g., 10%, 15%, 20%, 25%, 30% and 40%). FIGS. 21 and 22 provide representative dissolution profiles for morphine sulfate and oxycodone hydrochloride, respectively.

### Example 9

### Dissolution Testing of Various % Modified Release Coating Levels and Enteric Coating Levels

Two lots (˜3 kg) of morphine sulfate/oxycodone (3:2 by weight ratio) core pellets were coated using RS/RL polymer ratios of 90/10 (Lot 1, see Table 13) and 80/20 (Lot 2). Each lot was coated with different coating levels (25%, 35%, 45%, 50% and 55%) and samples were collected during the coating process. Dissolution testing (FIGS. 19 and 20) was performed on Lots 1 and 2 at the different coating levels.

In addition, coated pellets obtained from Lot 1 (at a 50% RS/RL coating level) were subjected to enteric coating at different % coating levels (10%, 15%, 25%, 30% and 40%) to produce enteric coated tablets and dissolution testing was performed (FIGS. 21 and 22).

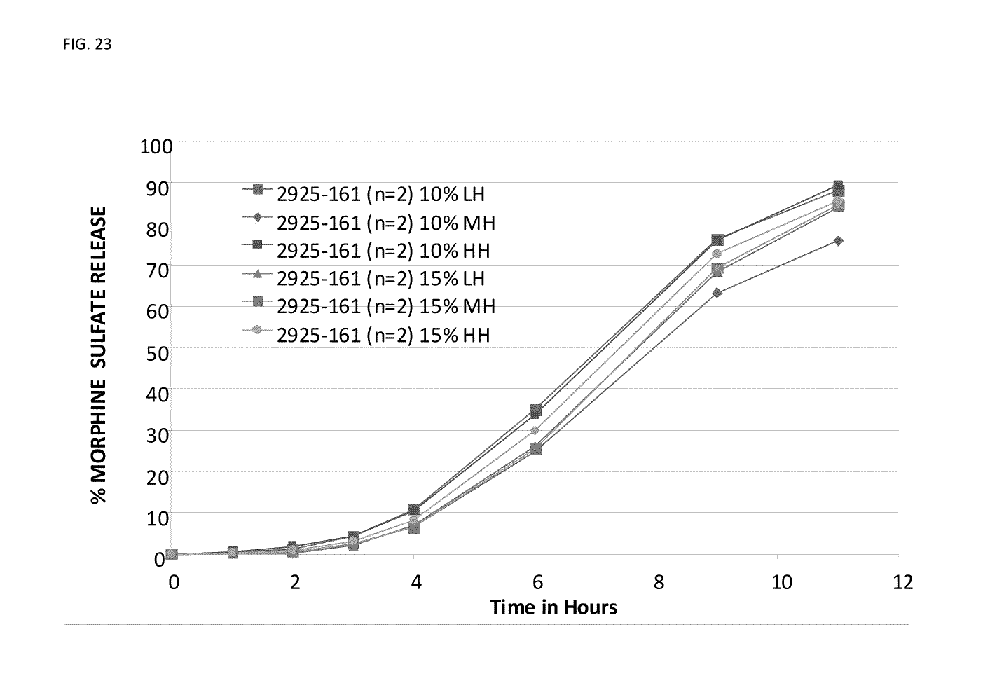

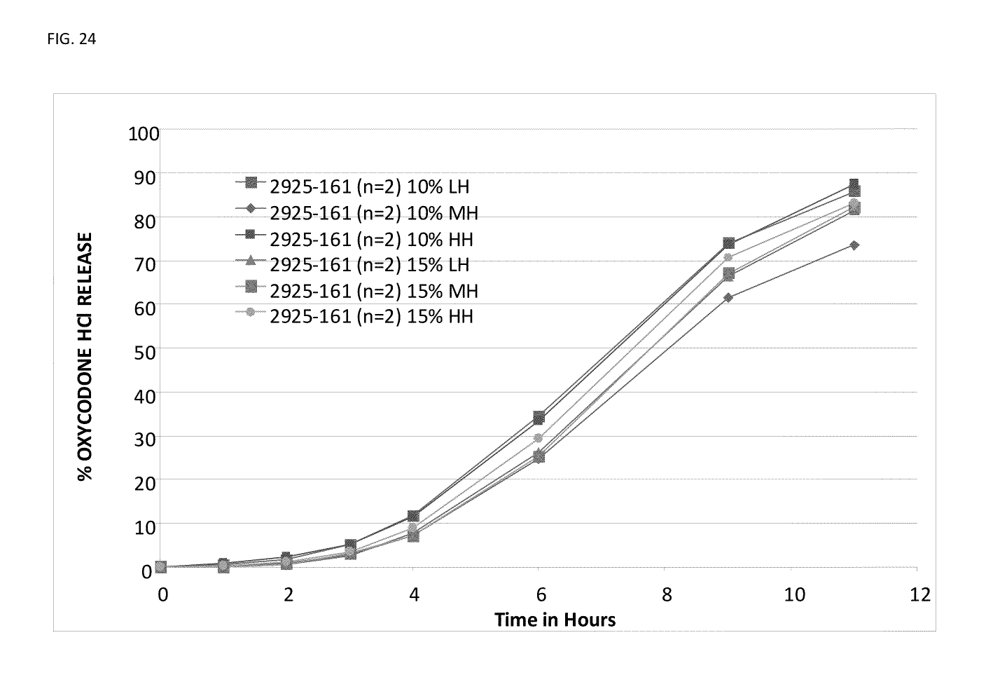

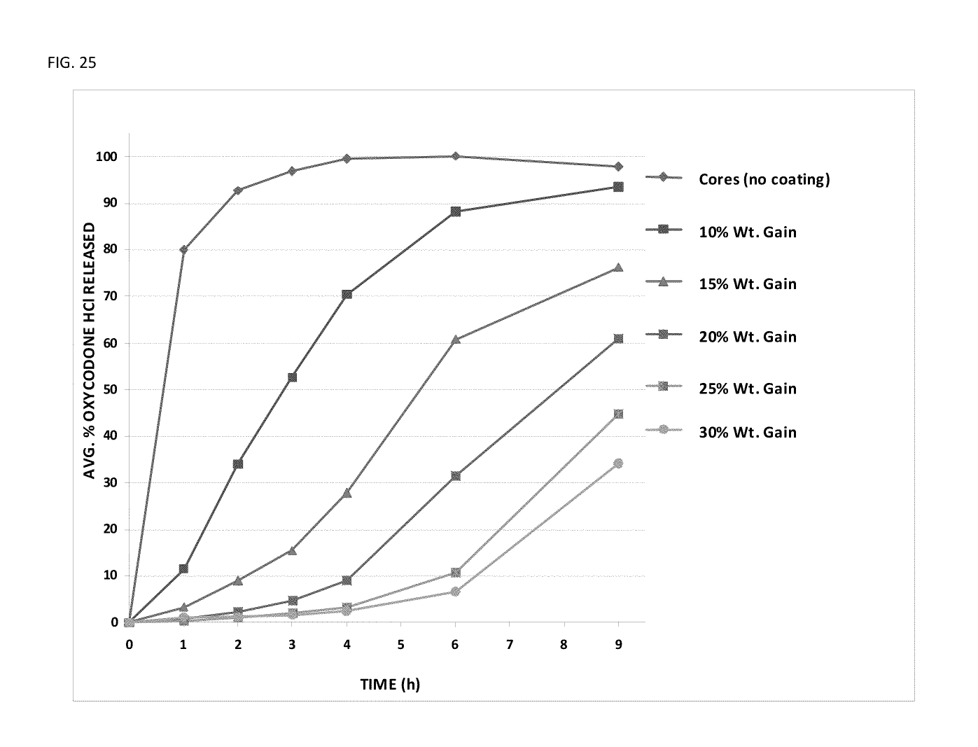

Enteric coated tablet lots (using 10% and 15% enteric coat) were also analyzed for dissolution as a function of tablet hardness (low, medium or high) to determine the resistance of the tablets to various compression levels (FIGS. 23 and 24).

A summary of the dissolution testing is provided in Table 15.

TABLE 15
Dissolution Testing Experiments.

| Test Performed | Batch # | Stage | Coating Level % |
|---|---|---|---|
| Dissolution | 2925-069 | Modified Release | 25 |
| | (RS/RL | Coated Beads | 35 |
| | 80/20) | | 45 |
| | | | 50 |
| | | | 55 |
| Dissolution | 2925-076 | Modified Release | 25 |
| | (RS/RL | Coated Beads | 35 |
| | 90/10) | | 45 |
| | | | 50 |
| | | | 55 |
| Dissolution | 2925-115 | Enteric Coated Tablets | 10 |
| | (RS/RL | (50% Modified Release | 15 |
| | 90/10) | Coated Beads) | 25 |
| | | | 30 |
| | | | 40 |
| Test Performed | Batch # | Stage | Tablet Hardness |
| Dissolution | 2925-161 | Tablet Compression | Low Hardness |
| | (RS/RL | (10% Enteric Coated | Mid Hardness |
| | 90/10) | Tablets) | High Hardness |
| Dissolution | 2925-161 | Tablet Compression | Low Hardness |
| | (RS/RL | (15% Enteric Coated | Mid Hardness |
| | 90/10) | Tablets) | High Hardness |

FIGS. 19 and 20 show the versatility of modified release core beadlets at various % coating levels in obtaining the dissolution profile of interest. A full spectrum of dissolution profiles allows for the targeting of specific in vivo pharmacokinetic plasma levels and the determination of in vitro to in vivo correlations.

FIGS. 21 and 22 also show the versatility of enteric coated, modified release core beadlets at various % enteric coating levels in obtaining the dissolution profile of interest. Once again, a full spectrum of dissolution profiles allows for the targeting of specific in vivo pharmacokinetic plasma levels and the determination of in vitro to in vivo correlations.

FIGS. 23 and 24 show the effect of compression forces on tablets that contain enteric coated beadlets comprising a modified release coated pellet of morphine sulfate and oxycodone hydrochloride. It is generally known that a high compression force can significantly reduce the dissolution of tablets, especially when coating polymers are employed that are known to be brittle, such as with ammonio methacrylate copolymer Type A and B. FIGS. 23 and 24 demonstrate that a low or high compression force does not affect the dissolution of tablets. This result is unexpected and demonstrates the resilience of the formulation/coatings to compression forces.

### Example 10

### Controlled Release Opioid Formulation Formulations

Following the procedure of Example 6, the following formulations were prepared:

TABLE 16
Tablet Formulations of Modified Release Beadlets (RS/RL) with/without
Enteric Coating (Eudragit L100-55 Type C).

| | Modified | | | | | |
|---|---|---|---|---|---|---|
| | Release | | Modified Release | | | |
| | RS/RL 85/15 | | RS/RL 80/20 | | | |
| | Modified Release Coating Level % | | | | | |
| | 10% | 15% | 10% | 20% | 10% | 20% |
| Component | % w/w | | | | | |
| Oxycodone Hydrochloride | 18.18 | 17.39 | 18.09 | 16.58 | 15.19 | 13.92 |
| Cellulose Microcrystalline | 68.18 | 65.22 | 63.32 | 58.04 | 56.96 | 52.22 |
| Povidone K30 | 3.64 | 3.48 | — | — | 3.04 | 2.79 |
| Hypromellose | — | — | 8.14 | 7.46 | — | — |
| (Methocel E15 Premium LV) | | | | | | |
| Polyoxyl 35 Castor Oil | 0.91 | 0.87 | 0.90 | 0.83 | 0.76 | 0.70 |
| Ammonio Methacrylate | 4.68 | 6.71 | 5.59 | 10.25 | 4.70 | 8.61 |
| Copolymer Type B (RS PO) | | | | | | |
| Ammonio Methacrylate | 0.83 | 1.18 | 1.40 | 2.56 | 1.17 | 2.15 |
| Copolymer Type A (RL PO) | | | | | | |
| Triethyl Citrate NF/EP | 0.86 | 1.24 | 0.70 | 1.28 | 0.59 | 1.08 |
| Magnesium Stearate 5712 | 2.73 | 3.91 | 1.36 | 2.49 | 1.14 | 2.09 |
| Enteric Coating | — | — | — | — | 9.68 | 9.68 |
| Eudragit L 100-55 Type C | | | | | | |
| Triethyl Citrate NF/EP | — | — | — | — | 0.97 | 0.97 |
| Talc (197 Grade) USP/EP/JP | — | — | 0.5 | 0.5 | 4.84 | 4.84 |
| Silicon Dioxide Colloidal | — | — | — | — | 0.96 | 0.96 |
| Total | 100 | 100 | 100 | 100 | 100 | 100 |

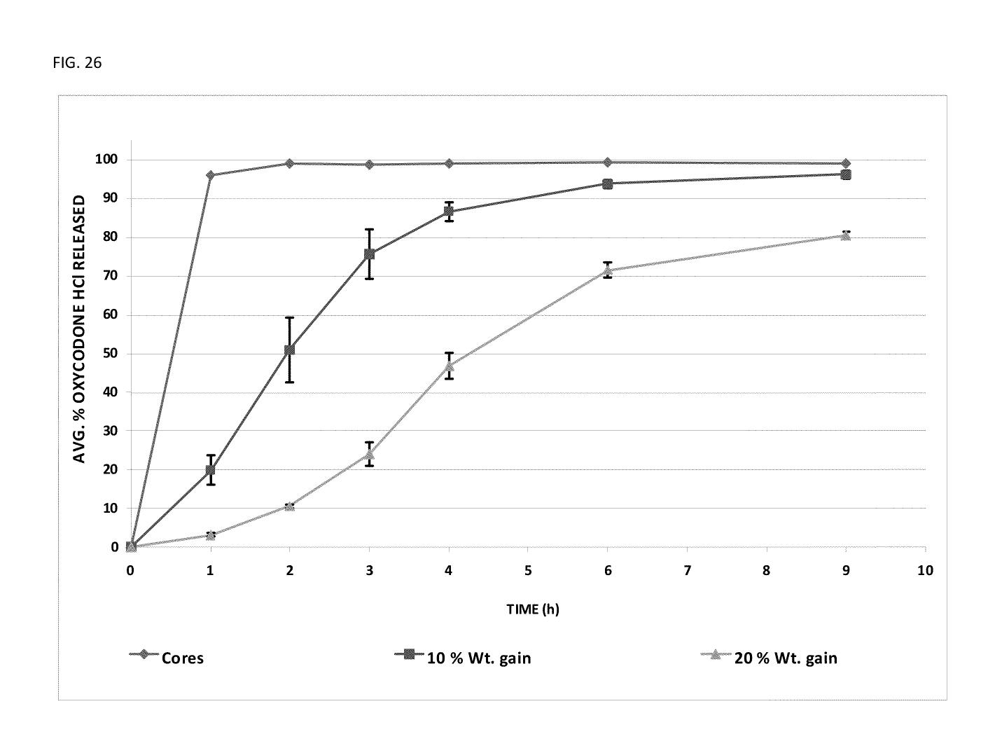

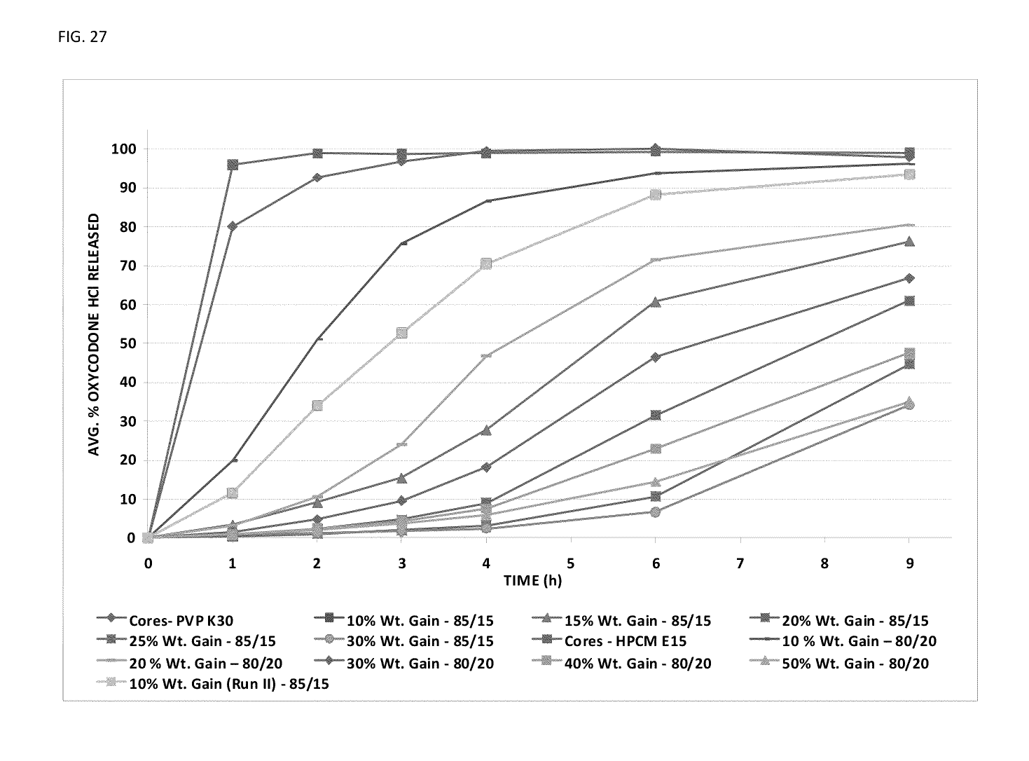

FIGS. 25-27 provide representative dissolution profiles for morphine sulfate and oxycodone hydrochloride, respectively, for the formulations provided in Table 16. These figures show the versatility of modified release core beadlets at various % coating levels in obtaining the dissolution profile of interest. Enteric coated beadlet formulations are also provided that allow for a full spectrum of dissolution profiles to be achieved.

### Example 11

### MoxDuo Controlled Release Formulations

Controlled release formulations containing 30 mg of morphine sulfate and 20 mg of oxycodone hydrochloride were prepared in the form of beadlets. (“MoxDuo CR beadlets (30:20 mg)”). The MoxDuo CR beadlets (30:20 mg) comprised a core, a coat, and an enteric coat as follows:

TABLE 17
Formulation of MoxDuo CR beadlets (30:20 mg).

| | | Test Product | |
|---|---|---|---|
| | | Quantity | |
| | | (mg) per | |
| | | unit dose | |
| Component | Function | of beadlets | % |

Beadlet Core

| | | | |
|---|---|---|---|
| Morphine sulfate | Drug substance | 30.0 | 16.7 |
| Oxycodone hydrochloride | Drug substance | 20.0 | 11.1 |
| Microcrystalline cellulose | Filler/diluent | 44.5 | 24.7 |
| Povidone K30 | Binder | 4.0 | 2.2 |
| Polyoxyl 35 castor oil | Lubricant | 1.5 | 0.8 |
| Purified water* | Process aid | — | — |
| Sub total | | 100.0 | 55.5 |

Beadlet Coat

| | | | |
|---|---|---|---|
| Ammonio methacrylate | Film forming agent | 3.07 | 1.7 |
| copolymer, Type A⁽¹⁾ | | | |
| Ammonio methacrylate | Film forming agent | 27.7 | 15.4 |
| copolymer, Type B⁽¹⁾ | | | |
| Triethyl citrate | Plasticizer | 3.84 | 2.1 |
| Talc (197) | Antitacking agent | 15.39 | 8.6 |
| Purified water* | Process aid | — | — |
| Sub total | | 50.0 | 27.8 |

Enteric Coat

| | | | |
|---|---|---|---|
| Methacrylic acid, copolymer | Film forming agent | 18.46 | 10.3 |
| dispersion Eudragit L30-D55(2) | | | |
| Triethyl citrate | Plasticizer | 2.31 | 1.3 |
| Talc (197) | Antitacking agent | 9.23 | 5.1 |
| Purified water* | Process aid | — | — |
| Sub total | | 30 | 16.7 |
| Total | | 180.0 | 100 |

*Removed during drying by evaporation
⁽¹⁾Expressed as the 30% solids, remaining water removed by evaporation

Tablets containing the MoxDuo CR beadlets (30:20 mg) along with a tamper and abuse deterrent feature (“MoxDuo CR tablets (30:20 mg)”) were also prepared. The MoxDuo CR tablets (30:20 mg) are comprised of a dry blend of (i) the MoxDuo CR beadlets (30:20 mg), (ii) multiparticulate hydrophilic polymer beadlets that comprise abuse deterrent attributes (“ADF beadlet blend”), and (iii) excipient ingredients, as follows:

TABLE 18
Formulation of MoxDuo CR tablets (30:20 mg).

| | | Test Product | |
|---|---|---|---|
| | | Quantity | |
| | | (mg) per | |
| Component | Function | tablet | % |
| MoxDuo CR beadlets | CR beadlet | 180 | 22.5 |
| (30:20 mg) | | | |
| ADF beadlet blend | Filler/diluent, | 180 | 22.5 |
| | abuse deterrent | | |
| | feature | | |
| Microcrystalline cellulose, PH102 | Filler/diluent | 317.2 | 39.7 |
| Microcrystalline cellulose, PH200 | Filler/diluent | 79.3 | 9.9 |
| Carbopol 971P | Hydrophilic | 18.1 | 2.3 |
| | polymer | | |
| Croscarmellose sodium | Disintegrant | 22.7 | 2.8 |
| Magnesium stearate | Lubricant | 2.7 | 0.3 |
| Total | | 800.0 | 100 |

The ADF beadlet blend comprises Carbopol beadlets as the abuse deterrent component (70%), and Meglumine beadlets as a pH modifier (30%). The formulations for the Carbopol beadlets and the Meglumine beadlets are as follows:

TABLE 19
Formulation of Carbopol beadlet.

| | | Test Product | |
|---|---|---|---|
| | | Quantity | |
| | | (mg) per | |
| Component | Function | batch | % |

Carbopol Beadlet Core

| | | | |
|---|---|---|---|
| Carbopol 971P | Hydrophilic | 300.0 | 8.3 |
| | polymer | | |
| Microcrystalline Cellulose, | Filler/diluent | 2700.0 | 74.5 |
| PH101 | | | |
| Purified water* | Process aid | — | — |
| Sub total | | 3000.0 | 82.8 |

Carbopol Beadlet Enteric Coat

| | | | |
|---|---|---|---|
| Opadry 20A19301 | Sealer | 150.0 | 4.1 |
| Methacrylic acid, copolymer | Film forming agent | 290.7 | 8.1 |
| dispersion, Eudragit L30-D55⁽¹⁾ | | | |
| Triethyl citrate | Plasticizer | 36.4 | 1.0 |
| Talc (197) | Antitacking agent | 145.4 | 4.0 |
| Purified water* | Process aid | — | — |
| Sub total | | 622.5 | 17.2 |
| Total | | 3622.5 | 100 |

*Removed during drying by evaporation
⁽¹⁾Expressed as the 30% solids, remaining water removed by evaporation

TABLE 20
Formulation of Meglumine beadlet.

| | | Test Product | |
|---|---|---|---|
| | | Quantity | |
| | | (mg) per | |
| Component | Function | batch | % |

Meglumine Beadlet Core

| | | | |
|---|---|---|---|
| Meglumine | pH modifier | 720.0 | 20.0 |
| Plasdone K-29/32 | Filler/diluent | 90.0 | 2.5 |
| Microcrystalline cellulose, | Filler/diluents | 2790.0 | 77.5 |
| PH101 | | | |
| Purified water* | Process aid | — | — |
| Sub total | | 3600.0 | 82.8 |

Meglumine Beadlet Enteric Coat

| | | | |
|---|---|---|---|
| Opadry 20A19301 | Sealer | 180 | 4.1 |
| Methacrylic acid, copolymer | Film forming agent | 350.2 | 8.1 |
| dispersion, Eudragit L30-D55⁽¹⁾ | | | |
| Triethyl citrate | Plasticizer | 43.6 | 1.0 |
| Talc (197) | Antitacking agent | 173.2 | 4.0 |
| Purified water* | Process aid | — | — |
| Sub total | | 747.0 | 17.2 |
| Total | | 4347.0 | 100 |

*Removed during drying by evaporation
⁽¹⁾Expressed as the 30% solids, remaining water removed by evaporation

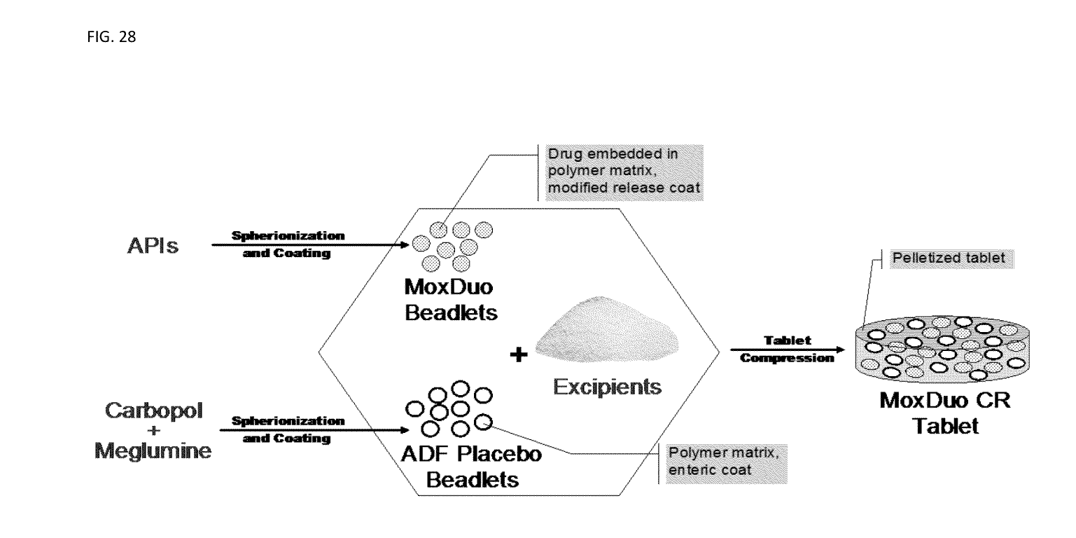

To prepare the MoxDuo CR tablets (30:20 mg), the dry blend is compressed into tablets using a standard, gravity-feed, pharmaceutical tableting machine, as shown in the FIG. 28.

### Example 12

### PK Profile Comparison Among MoxDuo CR beadlets (30:20 mg), MoxDuo CR tablets (30:20 mg), and MS Contin/OxyContin

The plasma drug concentration vs. time course profiles and pharmacokinetic parameters of MoxDuo CR beadlets (30:20 mg), MoxDuo CR tablets (30:20 mg), and MS Contin/OxyContin were assessed and compared in healthy adult humans.

Fourteen healthy adult subjects were administered the following three treatments using a single-dose, three-period, three-sequence, three-treatment crossover design:

(1) MoxDuo CR beadlets (30:20 mg), encapsulated for oral administration;

(2) MoxDuo CR tablets (30:20 mg);

(3) Co-administration of MS Contin 30 mg (morphine sulfate) and OxyContin 20 mg (oxycodone hydrochloride).

Each subject received one of the three treatments on days 1, 8, and 15 of the study, in which the order of the treatment was randomly chosen. Blood samples for drug concentration measurements were obtained within 10 minutes prior to each treatment, and at 0.5, 1, 2, 3, 4, 5, 5.5, 6, 6.5, 7, 8, 10, 12, 14, 18, 21, 24, 48, and 72 hours after treatment.

In addition, each subject received four 50-mg doses of oral naltrexone during each period of the study, the first to be administered approximately 12 hours prior to each study medication administration (days -1, 7, and 14), the second to be administered within 0.5 hours prior to each study medication administration (days 1, 8, and 15), the third to be administered within 0.5 hours prior to the 12-hour blood sampling (days 1, 8, and 15), and the fourth to be administered approximately 24 hours post dosing after the 24-hour PK blood sample is obtained.

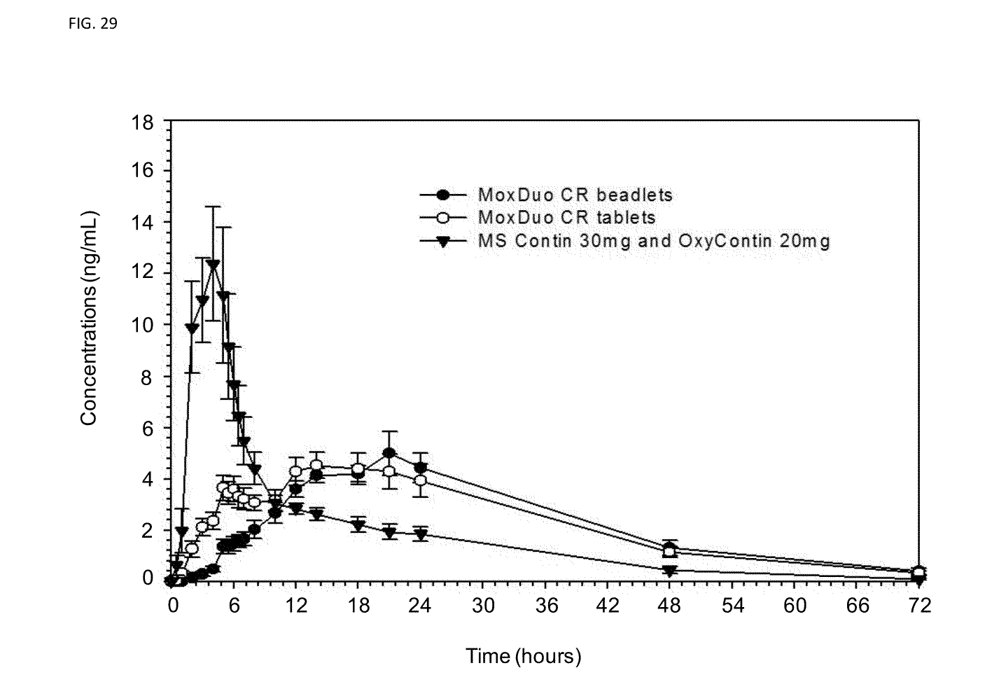

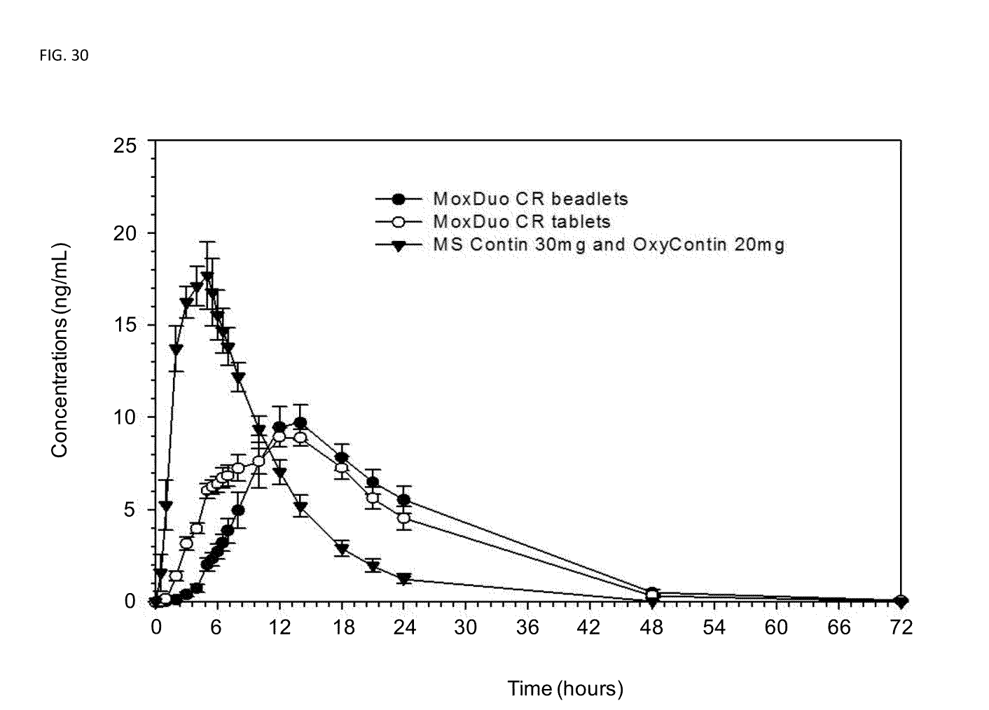

The results indicate that the morphine and oxycodone PK profiles for MoxDuo CR beadlets (30:20 mg) and MoxDuo CR tablet (30:20 mg) were similar, as shown in FIGS. 29 and 30 and in Table 19. These results suggest that the presence of the ADF beadlet blend in the MoxDuo CR tablet (30:20 mg) had little effect on the PK profile. However, compared to MoxDuo CR beadlets (30:20 mg) and MoxDuo CR tablet (30:20 mg), the morphine and oxycodone PK profiles for MS Contin/OxyContin were different, exhibiting mean morphine Cₘₐₓ that was over 160% greater and mean morphine tₘₐₓ that was over 80% less. These results are reflected in FIG. 29, which shows the substantial differences in mean morphine Cₘₐₓ and Tₘₐₓ between MoxDuo CR beadlets (30:20 mg) and MoxDuo CR tablet (30:20 mg), and MS Contin/OxyContin. Yet, the mean morphine AUCₜ for MoxDuo CR beadlets (30:20 mg) and MoxDuo CR tablet (30:20 mg) were greater than the morphine AUCₜ for MS Contin/OxyContin, which suggests that a greater amount of morphine was released by CR beadlets (30:20 mg) and MoxDuo CR tablet (30:20 mg) as compared to MS Contin/OxyContin, despite having a substantially smaller morphine Cₘₐₓ and greater Tₘₐₓ.

TABLE 21
Individual Morphine PK Metrics.

| | | | | |
|---|---|---|---|---|
| | AUCₜ | Cₘₐₓ | tₘₐₓ | t_{1/2} |
| Subject | (ng*h/mL) | (ng/mL) | (h) | (h) |

MoxDuo CR beadlets (30:20 mg)

| | | | | |
|---|---|---|---|---|
| 1 | 178.81 | 6.64 | 24.00 | NC |
| 2 | 124.26 | 4.20 | 14.00 | 13.41 |
| 3 | 177.06 | 5.34 | 14.00 | 16.91 |
| 4 | 264.90 | 9.58 | 21.00 | 19.09 |
| 5 | 158.25 | 5.47 | 18.00 | 13.70 |
| 6 | 213.93 | 10.25 | 21.00 | 11.52 |
| 7 | 122.16 | 4.86 | 14.00 | 14.78 |
| 8 | 78.99 | 3.34 | 14.00 | 12.21 |
| 9 | 146.43 | 4.86 | 24.00 | NC |
| 10 | 92.06 | 4.33 | 12.00 | 10.77 |
| Mean | 155.68 | 5.89 | 17.60 | 14.05 |
| SD | 56.22 | 2.30 | 4.58 | 2.81 |

MoxDuo CR tablets (30:20 mg)

| | | | | |
|---|---|---|---|---|
| 1 | 171.69 | 5.10 | 14.00 | 11.92 |
| 2 | 125.49 | 3.32 | 21.00 | 14.76 |
| 3 | 177.99 | 5.30 | 18.00 | 21.43 |
| 4 | 326.10 | 9.20 | 21.00 | 13.73 |
| 5 | 148.08 | 4.09 | 14.00 | 15.06 |
| 6 | 202.57 | 6.31 | 14.00 | 10.66 |
| 7 | 152.07 | 4.79 | 12.00 | 10.11 |
| 8 | 89.66 | 4.07 | 14.00 | 9.54 |
| 9 | 144.79 | 4.23 | 21.00 | 12.97 |
| 10 | 64.21 | 3.55 | 8.00 | 8.07 |
| Mean | 160.26 | 5.00 | 15.70 | 12.83 |
| SD | 71.19 | 1.72 | 4.40 | 3.80 |

MS Contin/OxyContin

| | | | | |
|---|---|---|---|---|
| 1 | 140.40 | 15.76 | 2.00 | 11.39 |
| 2 | 111.21 | 10.23 | 4.00 | 11.02 |
| 3 | 135.89 | 16.78 | 2.00 | 15.04 |
| 4 | 252.98 | 32.33 | 5.00 | 14.02 |
| 5 | 122.38 | 11.93 | 2.00 | 15.66 |
| 6 | 140.38 | 19.40 | 3.00 | 12.34 |
| 7 | 140.63 | 17.96 | 3.00 | 9.99 |
| 8 | 57.61 | 8.28 | 1.00 | 8.96 |
| 9 | 98.49 | 11.88 | 3.00 | 9.44 |
| 10 | 80.77 | 10.28 | 2.00 | 6.20 |
| Mean | 128.07 | 15.48 | 2.70 | 11.41 |
| SD | 52.19 | 6.99 | 1.16 | 2.94 |

AUCₜ = total exposure to the final measurable concentration
Cₘₐₓ = peak exposure
Tₘₐₓ = time to peak exposure
t_{1/2} = apparent terminal half-life

TABLE 22
Individual Oxycodone PK Metrics.

| | | | | |
|---|---|---|---|---|
| | AUCₜ | Cₘₐₓ | tₘₐₓ | t_{1/2} |
| Subject | (ng*h/mL) | (ng/mL) | (h) | (h) |

MoxDuo CR beadlets (30:20 mg)

| | | | | |
|---|---|---|---|---|
| 1 | 246.40 | 8.97 | 24.00 | NC |
| 2 | 304.50 | 14.53 | 14.00 | 6.79 |
| 3 | 189.83 | 11.53 | 12.00 | 9.53 |
| 4 | 216.83 | 8.22 | 24.00 | NC |
| 5 | 158.78 | 9.26 | 12.00 | 4.53 |
| 6 | 222.51 | 9.87 | 14.00 | 5.08 |
| 7 | 174.58 | 9.68 | 12.00 | 6.91 |
| 8 | 163.76 | 9.31 | 14.00 | 4.39 |
| 9 | 247.51 | 12.44 | 14.00 | 7.16 |
| 10 | 225.70 | 17.56 | 10.00 | 7.63 |
| Mean | 215.04 | 11.14 | 15.00 | 6.50 |
| SD | 45.07 | 2.95 | 4.92 | 1.75 |

MoxDuo CR beadlets (30:20 mg)

| | | | | |
|---|---|---|---|---|
| 1 | 234.04 | 10.75 | 14.00 | 6.39 |
| 2 | 271.65 | 8.92 | 21.00 | 7.52 |
| 3 | 185.51 | 7.51 | 14.00 | 9.96 |
| 4 | 295.65 | 11.21 | 14.00 | 9.07 |
| 5 | 160.92 | 7.57 | 14.00 | 6.32 |
| 6 | 230.68 | 10.13 | 14.00 | 5.31 |
| 7 | 126.65 | 9.38 | 12.00 | 6.49 |
| 8 | 133.93 | 10.55 | 12.00 | 6.06 |
| 9 | 206.52 | 9.53 | 14.00 | 7.15 |
| 10 | 181.73 | 12.91 | 8.00 | 7.85 |
| Mean | 202.73 | 9.85 | 13.70 | 7.21 |
| SD | 55.95 | 1.65 | 3.20 | 1.43 |

MS Contin/OxyContin

| | | | | |
|---|---|---|---|---|
| 1 | 200.24 | 22.66 | 5.00 | 4.75 |
| 2 | 285.68 | 19.08 | 3.00 | 5.45 |
| 3 | 144.42 | 17.47 | 2.00 | 3.69 |
| 4 | 241.05 | 30.00 | 5.50 | 5.10 |
| 5 | 182.29 | 15.57 | 2.00 | 5.21 |
| 6 | 189.66 | 19.26 | 2.00 | 4.88 |
| 7 | 171.12 | 18.17 | 5.00 | 3.46 |
| 8 | 134.42 | 13.15 | 3.00 | 4.47 |
| 9 | 172.83 | 21.33 | 4.00 | 4.30 |
| 10 | 203.94 | 19.65 | 5.00 | 7.17 |
| Mean | 192.57 | 19.64 | 3.65 | 4.85 |
| SD | 44.56 | 4.54 | 1.42 | 1.04 |

AUCₜ = total exposure to the final measurable concentration
Cₘₐₓ = peak exposure
Tₘₐₓ = time to peak exposure
t_{1/2} = apparent terminal half-life

Similarly, compared to MoxDuo CR beadlets (30:20 mg) and MoxDuo CR tablet (30:20 mg), the mean oxycodone Cₘₐₓ for MS Contin/OxyContin was over 75% greater and the mean oxycodone tₘₐₓ for MS Contin/OxyContin was over 70% less, despite that MoxDuo CR beadlets (30:20 mg) and MoxDuo CR tablet (30:20 mg) exhibited a greater mean oxycodone AUC_{τ}. These results are depicted in FIG. 30.

Therefore, these results demonstrate that the MoxDuo CR beadlets (30:20 mg) and MoxDuo CR tablet (30:20 mg) exhibit a pharmacokinetic profile reflecting release of more morphine and oxycodone over a longer duration as compared to MS Contin/OxyContin.

### Example 13

### Steady-State Pharmacokinetics of MoxDuo CR tablets (30:20 mg)

MoxDuo CR tablets (30:20 mg) were assessed to characterize steady-state pharmacokinetics of morphine and oxycodone during repetitive (twice daily) administration of MoxDuo CR tablets (30:20 mg) to steady state.

Nine healthy adult subjects received a single MoxDuo CR tablet (30:20 mg) on days 1 and 8. Beginning on the morning of day 15, the subjects received a single MoxDuo CR tablet (30:20 mg) every 12 hours through the morning of day 20 (11 doses). Blood samples for drug concentration measurements were obtained within 10 minutes prior to each treatment on days 15 through 20. On day 20, blood samples were obtained at 0.5, 1, 2, 3, 4, 5, 6, 7, 8, 10, and 12 hours after the final treatment in the morning.

Each subject also received oral naltrexone (50 mg) approximately 12 hours prior to each study medication dosing (days −1, 7, and 14), approximately 0.5 hours prior to each dosing (days 1, 8, and 15-20), approximately 0.5 hour prior to the 12-hour blood sampling (days 1 and 8), and approximately 12 hours and 24 hours after the final dosing.

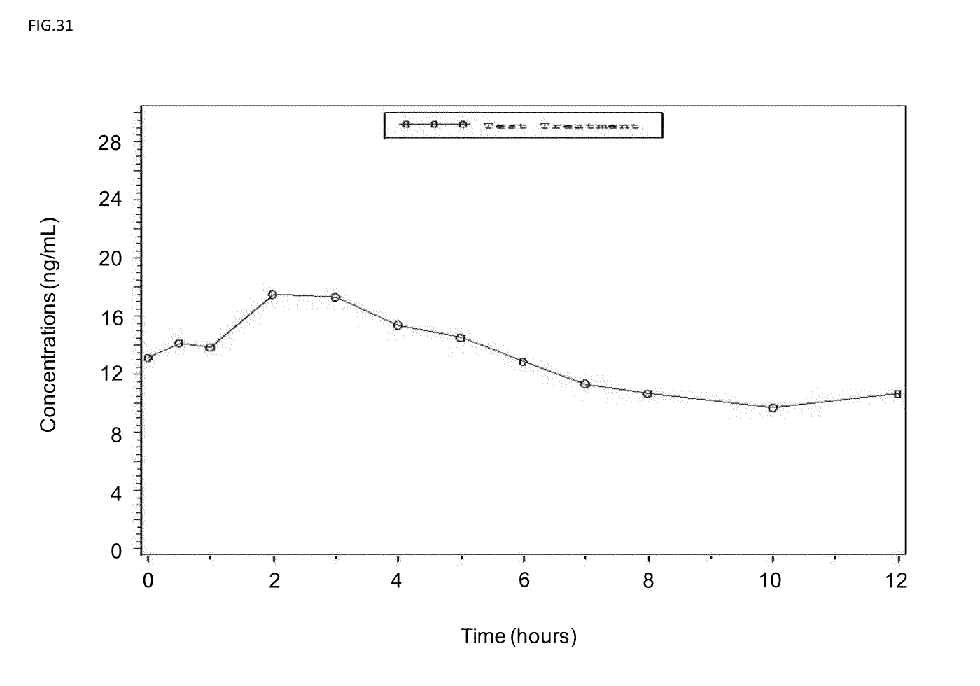

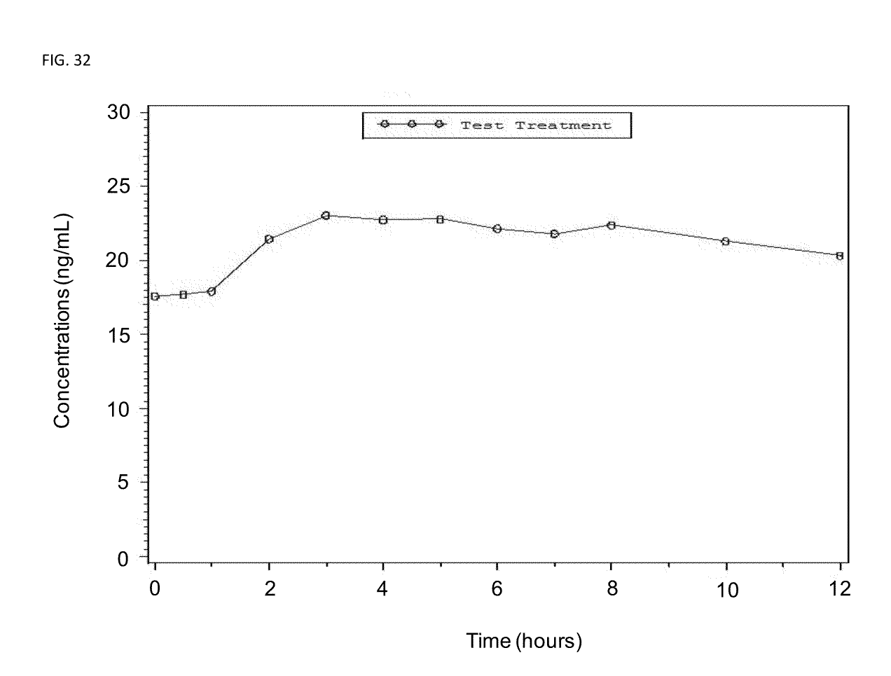

The results are shown in Table 23 and in FIGS. 31 (morphine) and 32 (oxycodone).

TABLE 23
Individual Morphine and Oxycodone PK Metrics at Steady State.

| | | | | | | |
|---|---|---|---|---|---|---|
| | | | | | | Fluctu- |
| | AUC_{ss,τ} | C_{ss,max} | C_{ss,min} | C_{ss,avg} | t_{ss,max} | ation |
| Subject | (ng * h/mL) | (ng/mL) | (ng/mL | (ng/mL) | (h) | (%) |

Morphine

| | | | | | | |
|---|---|---|---|---|---|---|
| 1 | 181.90 | 21.22 | 10.51 | 15.16 | 2.00 | 70.65 |
| 2 | 147.80 | 17.69 | 10.17 | 12.32 | 2.00 | 61.05 |
| 3 | 195.24 | 21.32 | 12.28 | 16.27 | 3.00 | 55.56 |
| 4 | 142.92 | 15.22 | 7.98 | 11.91 | 3.00 | 60.79 |
| 5 | 121.84 | 15.72 | 6.86 | 10.15 | 2.00 | 87.26 |
| 6 | 163.84 | 17.55 | 11.95 | 13.65 | 2.00 | 41.01 |
| 7 | 151.61 | 16.46 | 9.47 | 12.63 | 4.00 | 55.33 |
| 8 | 201.85 | 24.86 | 12.14 | 16.82 | 3.00 | 75.62 |
| 9 | 94.90 | 12.18 | 5.58 | 7.91 | 2.00 | 83.46 |
| Mean | 155.77 | 18.02 | 9.66 | 12.98 | 2.56 | 65.64 |
| SD | 34.52 | 3.84 | 2.41 | 2.88 | 0.73 | 14.87 |

Oxycodone

| | | | | | | |
|---|---|---|---|---|---|---|
| 1 | 463.47 | 42.59 | 35.65 | 38.62 | 3.00 | 17.98 |
| 2 | 239.35 | 21.77 | 16.55 | 19.95 | 3.00 | 26.17 |
| 3 | 287.88 | 26.50 | 19.69 | 23.99 | 8.00 | 28.35 |
| 4 | 182.80 | 17.49 | 10.30 | 15.23 | 6.00 | 47.20 |
| 5 | 218.92 | 19.38 | 13.20 | 18.24 | 5.00 | 33.85 |
| 6 | 228.42 | 21.65 | 13.44 | 19.04 | 6.00 | 43.15 |
| 7 | 227.60 | 21.08 | 14.52 | 18.97 | 4.00 | 34.59 |
| 8 | 269.36 | 24.86 | 16.21 | 22.45 | 4.00 | 38.57 |
| 9 | 197.31 | 19.31 | 14.69 | 16.44 | 3.00 | 28.12 |
| Mean | 257.24 | 23.85 | 17.14 | 21.44 | 4.67 | 33.11 |
| SD | 83.85 | 7.56 | 7.41 | 6.99 | 1.73 | 9.06 |

AUC_{ss,τ} = area under the concentration time course curve within a single dosing interval at steady state, where τ is the width of the dosing interval (AUC from the morning dose to 12 hours on day 20)
C_{ss,max} = maximum concentration at steady state peak exposure
C_{ss,min} = maximum concentration at steady state peak exposure
C_{ss,avg} = average concentration at steady-state, estimated as AUC_{ss,t} divided by τ
Tₘₐₓ = time to peak exposure
SD = standard deviation

The fluctuation index, calculated by dividing the difference between C_{ss,max} and C_{ss,min} with C_{ss,avg}, is 65.64% for morphine and 33.11% for oxycodone. These data suggest that, at steady state, the MoxDuo CR tablet (30:20 mg) can provide an even plasma concentration of both morphine and oxycodone without large fluctuations.

It should be understood, of course, that the foregoing relates only to certain disclosed embodiments of the present invention and that numerous modifications or alterations may be made therein without departing from the spirit and scope of the invention as set forth in the appended claims.

What is claimed is:

1. A pharmaceutical formulation for treatment of pain in a human, comprising a controlled release component, wherein:
(a) the controlled release component comprises one or more cores;
(b) the controlled release component comprises oxycodone;
(c) the pharmaceutical formulation comprises a steady state plasma concentration profile of the oxycodone having a fluctuation index of about 90% or less; and
(d) the pharmaceutical formulation, when containing a total dose of about 20 mg of oxycodone and administered at a dosing interval of 12 hours, will produce an AUC_{ss,τ} that is about 100 ng*h/mL to about 550 ng*h/mL.

2. The pharmaceutical formulation of claim 1, wherein when the pharmaceutical formulation contains about 20 mg of oxycodone and administered at a dosing interval of 12 hours, C_{ss,max} of oxycodone is about 10 ng/mL to about 50 ng/mL.

3. The pharmaceutical formulation of claim 1, wherein when the pharmaceutical formulation contains about 20 mg of oxycodone and administered at a dosing interval of 12 hours, t_{ss,max} of oxycodone is about 1 hour to about 10 hours.

4. The pharmaceutical formulation of claim 1, wherein when the pharmaceutical formulation contains a different total dose of oxycodone than about 20 mg, AUC_{ss,τ} of oxycodone at the different total dose is proportional to AUC_{ss,τ} of oxycodone at 20 mg.

5. The pharmaceutical formulation of claim 2, wherein when the pharmaceutical formulation contains a different total dose of oxycodone than about 20 mg, C_{ss,max} of oxycodone at the different total dose is proportional to C_{ss,max} of oxycodone at 20 mg.

6. The pharmaceutical formulation of claim 1, wherein the controlled release component further comprises an opioid selected from the group consisting of morphine, codeine, hydromorphone, hydrocodone, dihydrocodeine, dihydromorphine, oxymorphone, mixtures thereof, and salts thereof.

7. The pharmaceutical formulation of claim 6, wherein the controlled release component comprises morphine or salts thereof.

8. The pharmaceutical formulation of claim 7, wherein the morphine is in the form of morphine sulfate and the oxycodone is in the form of oxycodone hydrochloride.

9. The pharmaceutical formulation of claim 1, in the form of a tablet or capsule.

10. The pharmaceutical formulation of claim 9, wherein the form is a tablet.

11. The pharmaceutical formulation of claim 1, wherein the controlled release component is in a form selected from the group consisting of pellets, beads, beadlets, granules, powder, or a combination thereof.

12. The pharmaceutical formulation of claim 11, wherein the controlled release component is in the form of a beadlet.

13. The pharmaceutical formulation of claim 1, wherein the pharmaceutical formulation further comprises an abuse deterrent component.

14. The pharmaceutical formulation of claim 13, wherein the abuse deterrent component comprises a core comprising one or more materials that are both hydrophilic and hydrophobic.

15. The pharmaceutical formulation of claim 14, wherein the material that is both hydrophilic and hydrophobic is acrylic acid cross-linked with allyl ethers of polyalcohols.

16. The pharmaceutical formulation of claim 15, wherein the acrylic acid cross-linked with allyl ethers of polyalcohols is a carbomer.

17. The pharmaceutical formulation of claim 13, wherein the abuse deterrent component comprises a coating.

18. The pharmaceutical formulation of claim 13, wherein the abuse deterrent component further comprises an alkalizing agent.

19. The pharmaceutical formulation of claim 18, wherein the alkalizing agent is meglumine.

20. The pharmaceutical formulation of claim 13, wherein the abuse deterrent component is in a form selected from the group consisting of pellets, beads, beadlets, granules, powder, or a combination thereof.

21. The pharmaceutical formulation of claim 20, wherein the abuse deterrent component is in the form of a beadlet.

22. The pharmaceutical formulation of claim 1, further comprising one or more fillers or diluents, one or more hydrophilic polymers, one or more disintegrants, and one or more lubricants.

23. The pharmaceutical formulation of claim 22, wherein the one or more fillers or diluents comprises microcrystalline cellulose.

24. The pharmaceutical formulation of claim 22, wherein the one or more hydrophilic polymers comprises a carbomer.

25. The pharmaceutical formulation of claim 22, wherein the one or more disintegrants comprises croscarmellose sodium.

26. The pharmaceutical formulation of claim 22, wherein the one or more lubricants comprises magnesium stearate.

27. A pharmaceutical formulation for treatment of pain in a human, comprising a controlled release component, wherein:
(a) the controlled release component comprises one or more cores;
(b) the controlled release component comprises oxycodone; and
(c) the pharmaceutical formulation, when containing a total dose of about 20 mg of oxycodone, Cₘₐₓ is about 5 ng/mL to about 15 ng/mL following a single administration of the pharmaceutical formulation.

28. The pharmaceutical formulation of claim 27, wherein when the pharmaceutical formulation contains about 20 mg of oxycodone, tₘₐₓ of oxycodone is about 4 hours to about 24 hours following a single administration of the pharmaceutical formulation.

29. The pharmaceutical formulation of claim 27, wherein when the pharmaceutical formulation contains about 20 mg of oxycodone, AUCₜ of oxycodone is about 70 ng*h/mL to about 352 ng*h/mL following a single administration of the pharmaceutical formulation.

30. The pharmaceutical formulation of claim 27, wherein when the pharmaceutical formulation contains a different total dose of oxycodone than about 20 mg, Cₘₐₓ of oxycodone at the different total dose is proportional to Cₘₐₓ of oxycodone at 20 mg.

31. The pharmaceutical formulation of claim 29, wherein when the pharmaceutical formulation contains a different total dose of oxycodone than about 20 mg, AUCₜ of oxycodone at the different total dose is proportional to AUCₜ of oxycodone at 20 mg.

32. The pharmaceutical formulation of claim 27, wherein the controlled release component further comprises an opioid selected from the group consisting of morphine, codeine, hydromorphone, hydrocodone, dihydrocodeine, dihydromorphine, oxymorphone, mixtures thereof, and salts thereof.

33. The pharmaceutical formulation of claim 32, wherein the controlled release component comprises morphine or salts thereof.

34. The pharmaceutical formulation of claim 33, wherein the morphine is in the form of morphine sulfate and the oxycodone is in the form of oxycodone hydrochloride.

35. The pharmaceutical formulation of claim 27, in the form of a tablet or capsule.

36. The pharmaceutical formulation of claim 35, wherein the form is a tablet.

37. The pharmaceutical formulation of claim 27, wherein the controlled release component is in a form selected from the group consisting of pellets, beads, beadlets, granules, powder, or a combination thereof.

38. The pharmaceutical formulation of claim 37, wherein the controlled release component is in the form of a beadlet.

39. The pharmaceutical formulation of claim 27, wherein the pharmaceutical formulation further comprises an abuse deterrent component.

40. The pharmaceutical formulation of claim 39, wherein the abuse deterrent component comprises a core comprising one or more materials that are both hydrophilic and hydrophobic.

41. The pharmaceutical formulation of claim 40, wherein the material that is both hydrophilic and hydrophobic is acrylic acid cross-linked with allyl ethers of polyalcohols.

42. The pharmaceutical formulation of claim 41, wherein the acrylic acid cross-linked with allyl ethers of polyalcohols is a carbomer.

43. The pharmaceutical formulation of claim 39, wherein the abuse deterrent component comprises a coating.

44. The pharmaceutical formulation of claim 39, wherein the abuse deterrent component further comprises an alkalizing agent.

45. The pharmaceutical formulation of claim 44, wherein the alkalizing agent is meglumine.

46. The pharmaceutical formulation of claim 39, wherein the abuse deterrent component is in a form selected from the group consisting of pellets, beads, beadlets, granules, powder, or a combination thereof.

47. The pharmaceutical formulation of 46, wherein the abuse deterrent component is in the form of a beadlet.

48. The pharmaceutical formulation of claim 27, further comprising one or more fillers or diluents, one or more hydrophilic polymers, one or more disintegrants, and one or more lubricants.

49. The pharmaceutical formulation of claim 48, wherein the one or more fillers or diluents comprises microcrystalline cellulose.

50. The pharmaceutical formulation of claim 48, wherein the one or more hydrophilic polymers comprises a carbomer.

51. The pharmaceutical formulation of claim 48, wherein the one or more disintegrants comprises croscarmellose sodium.

52. The pharmaceutical formulation of claim 48, wherein the one or more lubricants comprises magnesium stearate.

53. A pharmaceutical formulation for treatment of pain in a human, comprising a controlled release component, wherein:
(a) the controlled release component comprises one or more cores;
(b) the controlled release component comprises morphine;
(c) the pharmaceutical formulation comprises a steady state plasma concentration profile of the morphine having a fluctuation index of about 90% or less; and
(d) the pharmaceutical formulation, when containing a total dose of about 30 mg of morphine and administered at a dosing interval of 12 hours, will produce an AUC_{ss,τ} that is about 60 ng*h/mL to about 240 ng*h/mL.

54. The pharmaceutical formulation of claim 53, wherein when the pharmaceutical formulation contains about 30 mg of morphine and administered at a dosing interval of 12 hours, C_{ss,max} of morphine is about 8 ng/mL to about 29 ng/mL.

55. The pharmaceutical formulation of claim 53, wherein when the pharmaceutical formulation contains about 30 mg of morphine and administered at a dosing interval of 12 hours, t_{ss,max} of morphine is about 1 hour to about 5 hours.

56. The pharmaceutical formulation of claim 53, wherein when the pharmaceutical formulation contains a different total dose of morphine than about 30 mg, AUC_{ss,τ} of morphine at the different total dose is proportional to AUC_{ss,τ} of morphine at 30 mg.

57. The pharmaceutical formulation of claim 54, wherein when the pharmaceutical formulation contains a different total dose of morphine than about 30 mg, C_{ss,max} of morphine at the different total dose is proportional to C_{ss,max} of morphine at 30 mg.

58. The pharmaceutical formulation of claim 53, wherein the controlled release component further comprises an opioid selected from the group consisting of oxycodone, codeine, hydromorphone, hydrocodone, dihydrocodeine, dihydromorphine, oxymorphone, mixtures thereof, and salts thereof.

59. The pharmaceutical formulation of claim 58, wherein the controlled release component comprises oxycodone or salts thereof.

60. The pharmaceutical formulation of claim 59, wherein the oxycodone is in the form of oxycodone hydrochloride and the morphine is in the form of morphine sulfate.

61. The pharmaceutical formulation of claim 53, in the form of a tablet or capsule.

62. The pharmaceutical formulation of claim 61, wherein the form is a tablet.

63. The pharmaceutical formulation of claim 53, wherein the controlled release component is in a form selected from the group consisting of pellets, beads, beadlets, granules, powder, or a combination thereof.

64. The pharmaceutical formulation of claim 63, wherein the controlled release component is in the form of a beadlet.

65. The pharmaceutical formulation of claim 53, wherein the pharmaceutical formulation further comprises an abuse deterrent component.

66. The pharmaceutical formulation of claim 65, wherein the abuse deterrent component comprises a core comprising one or more materials that are both hydrophilic and hydrophobic.

67. The pharmaceutical formulation of claim 66, wherein the material that is both hydrophilic and hydrophobic is acrylic acid cross-linked with allyl ethers of polyalcohols.

68. The pharmaceutical formulation of claim 67, wherein the acrylic acid cross-linked with allyl ethers of polyalcohols is a carbomer.

69. The pharmaceutical formulation of claim 65, wherein the abuse deterrent component comprises a coating.

70. The pharmaceutical formulation of claim 65, wherein the abuse deterrent component further comprises an alkalizing agent.

71. The pharmaceutical formulation of claim 70, wherein the alkalizing agent is meglumine.

72. The pharmaceutical formulation of claim 65, wherein the abuse deterrent component is in a form selected from the group consisting of pellets, beads, beadlets, granules, powder, or a combination thereof.

73. The pharmaceutical formulation of 72, wherein the abuse deterrent component is in the form of a beadlet.

74. The pharmaceutical formulation of claim 53, further comprising one or more fillers or diluents, one or more hydrophilic polymers, one or more disintegrants, and one or more lubricants.

75. The pharmaceutical formulation of claim 74, wherein the one or more fillers or diluents comprises microcrystalline cellulose.

76. The pharmaceutical formulation of claim 74, wherein the one or more hydrophilic polymers comprises a carbomer.

77. The pharmaceutical formulation of claim 74, wherein the one or more disintegrants comprises croscarmellose sodium.

78. The pharmaceutical formulation of claim 74, wherein the one or more lubricants comprises magnesium stearate.

79. A pharmaceutical formulation for treatment of pain in a human, comprising a controlled release component, wherein:
(a) the controlled release component comprises one or more cores;
(b) the controlled release component comprises morphine;
(c) the pharmaceutical formulation, when containing a total dose of about 30 mg of morphine, Cₘₐₓ is about 1 ng/mL to about 11 ng/mL following a single administration of the pharmaceutical formulation.

80. The pharmaceutical formulation of claim 79, wherein when the pharmaceutical formulation contains about 30 mg of morphine, tₘₐₓ of morphine is about 3 hours to about 25 hours following a single administration of the pharmaceutical formulation.

81. The pharmaceutical formulation of claim 79, wherein when the pharmaceutical formulation contains about 30 mg of morphine, AUCₜ of morphine is about 60 ng*h/mL to about 433 ng*h/mL following a single administration of the pharmaceutical formulation.

82. The pharmaceutical formulation of claim 79, wherein when the pharmaceutical formulation contains a different total dose of morphine than about 30 mg, Cₘₐₓ of morphine at the different total dose is proportional to Cₘₐₓ of morphine at 30 mg.

83. The pharmaceutical formulation of claim 79, wherein when the pharmaceutical formulation contains a different total dose of morphine than about 30 mg, AUCₜ of morphine at the different total dose is proportional to AUCₜ of morphine at 30 mg.

84. The pharmaceutical formulation of claim 79, wherein the controlled release component further comprises an opioid selected from the group consisting of oxycodone, codeine, hydromorphone, hydrocodone, dihydrocodeine, dihydromorphine, oxymorphone, mixtures thereof, and salts thereof.

85. The pharmaceutical formulation of claim 84, wherein the controlled release component comprises oxycodone or salts thereof.

86. The pharmaceutical formulation of claim 85, wherein the oxycodone is in the form of oxycodone hydrochloride and the morphine is in the form of morphine sulfate.

87. The pharmaceutical formulation of claim 79, in the form of a tablet or capsule.

88. The pharmaceutical formulation of claim 87, wherein the form is a tablet.

89. The pharmaceutical formulation of claim 79, wherein the controlled release component is in a form selected from the group consisting of pellets, beads, beadlets, granules, powder, or a combination thereof.

90. The pharmaceutical formulation of claim 89, wherein the controlled release component is in the form of a beadlet.

91. The pharmaceutical formulation of claim 79, wherein the pharmaceutical formulation further comprises an abuse deterrent component.

92. The pharmaceutical formulation of claim 91, wherein the abuse deterrent component comprises a core comprising one or more materials that are both hydrophilic and hydrophobic.

93. The pharmaceutical formulation of claim 92, wherein the material that is both hydrophilic and hydrophobic is acrylic acid cross-linked with allyl ethers of polyalcohols.

94. The pharmaceutical formulation of claim 93, wherein the acrylic acid cross-linked with allyl ethers of polyalcohols is a carbomer.

95. The pharmaceutical formulation of claim 91, wherein the abuse deterrent component comprises a coating.

96. The pharmaceutical formulation of claim 91, wherein the abuse deterrent component further comprises an alkalizing agent.

97. The pharmaceutical formulation of claim 96, wherein the alkalizing agent is meglumine.

98. The pharmaceutical formulation of claim 91, wherein the abuse deterrent component is in a form selected from the group consisting of pellets, beads, beadlets, granules, powder, or a combination thereof.

99. The pharmaceutical formulation of 98, wherein the abuse deterrent component is in the form of a beadlet.

100. The pharmaceutical formulation of claim 79, further comprising one or more fillers or diluents, one or more hydrophilic polymers, one or more disintegrants, and one or more lubricants.

101. The pharmaceutical formulation of claim 100, wherein the one or more fillers or diluents comprises microcrystalline cellulose.

102. The pharmaceutical formulation of claim 100, wherein the one or more hydrophilic polymers comprises a carbomer.

103. The pharmaceutical formulation of claim 100, wherein the one or more disintegrants comprises croscarmellose sodium.

104. The pharmaceutical formulation of claim 100, wherein the one or more lubricants comprises magnesium stearate.

105. A pharmaceutical formulation for treatment of pain in a human, comprising a controlled release component, wherein:
(a) the controlled release component comprises one or more cores;
(b) the controlled release component comprises oxycodone hydrochloride and morphine sulfate;
(c) the pharmaceutical formulation, when containing a total dose of about 20 mg of oxycodone hydrochloride, Cₘₐₓ of oxycodone is about 5 ng/mL to about 15 ng/mL following a single administration of the pharmaceutical formulation; and
(d) the pharmaceutical formulation, when containing a total dose of about 30 mg of morphine sulfate, Cₘₐₓ of morphine is about 1 ng/mL to about 11 ng/mL following a single administration of the pharmaceutical formulation.

106. The pharmaceutical formulation of claim 105, wherein when the pharmaceutical formulation contains about 20 mg of oxycodone hydrochloride and 30 mg of morphine sulfate, tₘₐₓ is about 4 hours to about 24 hours for oxycodone and about 3 hours to about 25 hours for morphine following a single administration of the pharmaceutical formulation.

107. The pharmaceutical formulation of claim 105, wherein when the pharmaceutical formulation contains about 20 mg of oxycodone hydrochloride and 30 mg of morphine sulfate, AUCₜ is about 70 ng*h/mL to about 352 ng*h/mL for oxycodone and about 60 ng*h/mL to about 433 ng*h/mL for morphine following a single administration of the pharmaceutical formulation.

108. The pharmaceutical formulation of claim 105, wherein the pharmaceutical formulation further comprises an abuse deterrent component.

109. A method of controlling release of one or more compounds having opioid receptor agonist activity for absorption in a human, wherein the method comprises administering the pharmaceutical formulation of claim 1, 27, 53, 79, or 105.

110. A method of treating pain in a human, comprising administering the pharmaceutical formulation of claim 1, 27, 53, 79, or 105.
